(19) United States
(12) Patent Application Publication
(10) Pub. No.: US 2007/0027093 A1
(43) Pub. Date: Feb. 1, 2007

(54) Anorectic

(75) Inventors: Nobuya Ogawa, Takatsuki (JP); Chihiro Okuma, Takatsuki (JP); Noboru Furukawa, Takatsuki (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Japan Tobacco Inc., Tokyo (JP)

(21) Appl. No.: 11/495,095

(22) Filed: Jul. 28, 2006

Related U.S. Application Data
(63) Continuation of application No. PCT/JP05/01643, filed on Jan. 28, 2005.
(60) Provisional application No. 60/598,037, filed on Aug. 2, 2004.

(30) Foreign Application Priority Data
Jan. 30, 2004 (JP) 2004-024812

(51) Int. Cl. A61K 31/70; A61K 31/365; A61K 31/542; A61K 31/5383; A61K 31/519; A61K 31/426; A61K 31/22; A61K 31/175

(52) U.S. Cl. 514/25; 514/230.5; 514/260.1; 514/224.2; 514/92; 514/423; 514/460; 514/548; 514/369; 514/635; 514/592

(57) ABSTRACT

The present invention relates to an anorectic containing a compound having a DGAT inhibitory activity (DGAT1 inhibitory activity) or a prodrug thereof or a pharmaceutically acceptable salt thereof as an active ingredient. The present invention provides an anti-obesity drug which is an anorectic that does not directly act on the central nervous system and is satisfactory in terms of activity, and a therapeutic strategy for preventing or treating obesity.

### TECHNICAL FIELD

The present invention relates to an anorectic action of a compound having a DGAT (diacylglycerol acyltransferase) inhibitory activity (e.g., DGAT1 inhibitory activity). Moreover, the present invention relates to a combined use of such DGAT inhibitors (e.g., DGAT1 inhibitor) and various drugs.

### BACKGROUND ART

It is known that various intracerebral neural activities and neurotransmitters are involved in the control of appetite in human and animals. These neural activities are affected by biochemical, neurological or endocrine signals that occur in the process of nutritive digestion, absorption, metabolism and storage.

Sugars and lipids themselves as nutrients, or metabolites in fat, muscule and liver cause biochemical signals that act promotively or suppressively on cerebral nerve activities involved in appetite.

It is also known that endocrine signals (e.g., CCK, GLP1, Enterostatin, ApoAIV etc.) or neural signals via chemical receptors of the gastrointestinal tract or from enteric plexus, during the process of digestion and absorption of sugars and lipids, affect gastrointestinal functions and cerebral nerve activities.

Moreover, it is known that fat tissue, which is a fat storage organ, produces endocrine or biochemical signals, such as leptin, adiponectin and free fatty acid, along with storage and consumption of fat. These signals alone or cooperative combinations of signals are considered to affect the central nervous system which controls appetite.

The DGAT1 inhibitor is expected to inhibit absorption of fat by suppressing re-synthesis of triglyceride in the gastrointestinal tract, and changes the above-mentioned signals that affect function of the gastrointestinal tract or brain.

In addition, the DGAT1 inhibitor is expected to change biochemical or endocrine signals from fat tissue by suppressing re-synthesis of triglyceride in the fat tissue.

Furthermore, it has been reported that DGAT1 deficient mice show an accelerated sensitivity of brain function to leptin which is an anti-obese factor derived from fat tissue. Therefore, a similar effect is expected by the administration of a DGAT1 inhibitor.

In the meantime, as a compound having a DGAT inhibitory activity, the following compounds are known.

The following compound has been disclosed to have a DGAT inhibitory activity (e.g., WO2004/47755, published after the priority date of the present application).

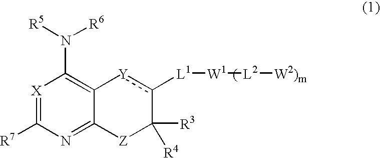

This reference discloses inhibition of DGAT. However, disclosure of anorectic action resulting from the inhibition of DGAT as in the present application is not contained at all.

For example, the following compound has been disclosed to have a DGAT inhibitory activity (e.g., JP-A-H5-213985).

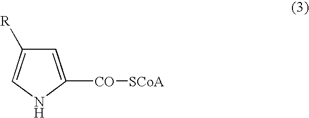

This reference discloses inhibition of ACAT and DGAT. However, disclosure of anorectic action resulting from the inhibition of DGAT as in the present application is not contained at all.

Similarly, the following compound has been disclosed to have a DGAT inhibitory activity (e.g., JP-A-H8-182496).

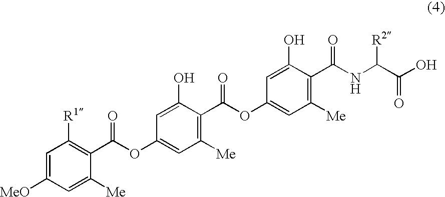

This reference discloses inhibition of DGAT. However, disclosure of anorectic action resulting from the inhibition of DGAT as in the present application is not contained at all.

Moreover, the following compound has been disclosed to have a DGAT inhibitory activity (e.g., WO00/58491).

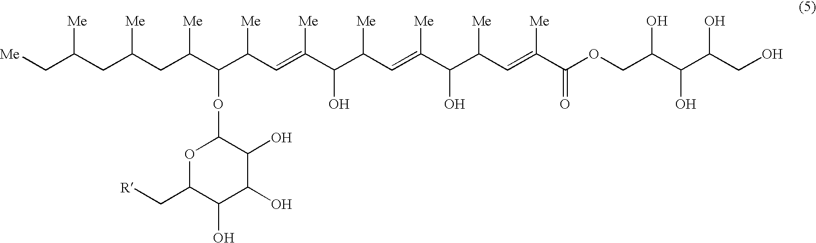

This reference discloses inhibition of DGAT. However, disclosure of anorectic action resulting from the inhibition of DGAT as in the present application is not contained at all.

Moreover, the following compound has been disclosed to have a DGAT inhibitory activity (e.g., JP-A-2004-67635).

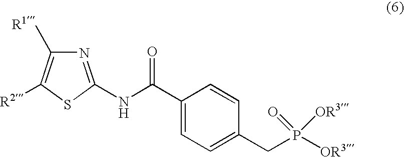

This reference discloses inhibition of DGAT. However, disclosure of anorectic action resulting from the inhibition of DGAT as in the present application is not contained at all.

As a compound having an anorectic action, ApoB secretion/MTP (Microsomal Triglyceride Transfer Protein) inhibitors have been disclosed (e.g., JP-A-2001-181209). As such compound, for example, the following formula has been disclosed. 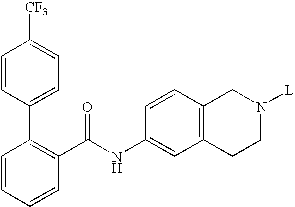
Specifically, the following compounds have been disclosed.

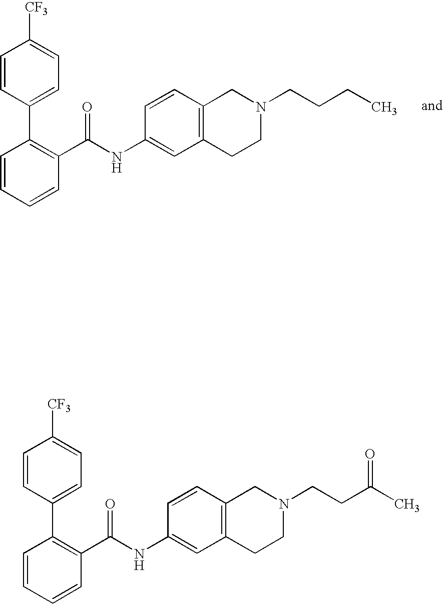

However, this reference does not disclose that these compounds have a DGAT inhibitory activity.

In addition, the reference discloses that similar compounds have been disclosed to have a suppressive action on fat absorption from small intestine, but does not disclose that these compounds have a DGAT inhibitory activity (e.g., JP-A-2001-172180).

While the development of anti-obesity drugs is currently ongoing, they are not satisfactory in terms of activity. The development of anorectic agents to prevent or treat obesity is also ongoing. However, since most of these anorectic agents directly act on the central nervous system and side effects are worried, the development of anorectic agents that do not directly act on the center has been strongly desired.

The problem to be solved by the present invention is provision of an anti-obesity drug which is an anorectic agent that does not directly act on the central nervous system and is satisfactory in terms of activity, and a therapeutic strategy for preventing or treating obesity.

### DISCLOSURE OF THE INVENTION

In view of the above-mentioned problem, the present inventors have intensively studied in an attempt to search a useful anorectic and surprisingly found that a compound having a DGAT inhibitory activity (e.g., DGAT1 inhibitory activity) has a remarkable anorectic activity, which resulted in the completion of the present invention.

More particularly, the invention provides the following [1]-[33].

[1] An anorectic comprising, as an active ingredient, a compound having a DGAT (diacylglycerol acyltransferase) inhibitory activity or a prodrug thereof or a pharmaceutically acceptable salt thereof.

[2] The anorectic of the above-mentioned [1], wherein the compound having a DGAT inhibitory activity is a compound represented by the following formula (1): 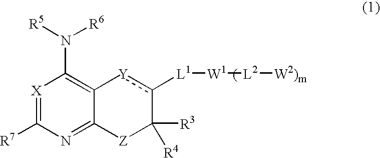
wherein
X is C(R¹) or N,
   wherein R¹ is a hydrogen atom, a C₁₋₈ alkyl group, a C₂₋₈ alkenyl group, a C₂₋₈ alkynyl group, a C₁₋₈ fluoroalkyl group, an aryl group, an aryl C₁₋₄ alkyl group, C(O)R^{a}, CO₂R^{a} or C(O)NR^{a}R^{b}, wherein R^{a} and R^{b} are the same or different and each is a hydrogen atom, a C₁₋₈ alkyl group, a C₂₋₈ alkenyl group, a C₂₋₈ alkynyl group, a C₁₋₈ fluoroalkyl group, an aryl group or an aryl C₁₋₄ alkyl group;
Y is C(R¹), C(R²)(R²), N or N(R²),
   wherein R¹ is as defined above and each R² is independently a hydrogen atom, a C₁₋₈ alkyl group, a C₂₋₈ alkenyl group, a C₂₋₈ alkynyl group, a C₁₋₈ fluoroalkyl group, C(O)R^{a}, CO₂R^{a}, C(O)NR^{a}R^{b}, an aryl group or an aryl C₁₋₄ alkyl group, wherein R^{a} and R^{b} are as defined above;
Z is O or S;
W¹ is an optionally substituted C₃₋₈ cycloalkylene group, an optionally substituted C₃₋₈ heterocycloalkylene group, an optionally substituted arylene group or an optionally substituted heteroarylene group;
W² is an optionally substituted C₃₋₈ cycloalkyl group, an optionally substituted C₃₋₈ heterocycloalkyl group, an optionally substituted aryl group or an optionally substituted heteroaryl group;
L¹ is a single bond, a C₁₋₄ alkylene group, a C₂₋₄ alkenylene group, O, C(O)N(R^{a}) or N(R^{a})C(O), wherein R^{a} is as defined above;
L² is a single bond, O, a C₁₋₄ alkylene group, a C₂₋₄ alkenylene group, a C₁₋₄ heteroalkylene group, C(O)N(R^{a}) or N(R^{a})C(O), wherein R^{a} is as defined above;
m is 0 or 1;
   when m is 1 and L² is a single bond, a substituent of W² may form, together with a substituent of W¹, a 5 to 7-membered ring that is condensed with W¹ and forms a fused ring or Spiro ring with W²;
R³ and R⁴
   are the same or different and each is a hydrogen atom, a C₁₋₈ alkyl group, a C₂₋₈ alkenyl group, a C₂₋₈ alkynyl group, C(O)R^{a}, CO₂R^{a}, C(O)NR^{a}R^{b} or a C₁₋₄ alkylene-OR^{a} group, wherein R^{a} and R^{b} are as defined above, or R³ and R⁴ may form a 3 to 6-membered ring together with the carbon atom binding thereto; or
R², R³ or R⁴
   may form, together with W¹, a 5 to 7-membered ring optionally having, in the ring, 1 to 3 heteroatoms selected from a nitrogen atom, an oxygen atom and a sulfur atom;
R⁵ and R⁶
   are the same or different and each is a hydrogen atom, a C₁₋₈ alkyl group, a C₂₋₈ alkenyl group, a C₂₋₈ alkynyl group, C(O)R^{a} or CO₂R^{a}, wherein R^{a} is as defined above, R⁵ and R⁶ may form an N-containing 5 to 7-membered ring together with the nitrogen atom binding thereto, or, when X is C(R¹), R⁵ or R⁶ may form, together with R¹, an N-containing 5 to 7-membered ring, wherein N may be substituted by R⁵ or R⁶;
R⁷ is a hydrogen atom, a C₁₋₈ alkyl group, a C₁₋₄ haloalkyl group, a C₂₋₈ alkenyl group, a C₂₋₈ alkynyl group, C(O)R^{a}, OR^{a} or NR^{a}R^{b}, wherein R^{a} and R^{b} are as defined above, or, when X is C(R¹), R⁷ may form, together with R¹, a 5 to 7-membered ring; and [symbol]
   is a single bond or a double bond;
   provided that the following compound is excluded: 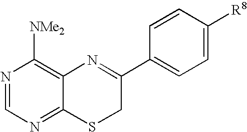
   wherein R⁸ is a hydrogen atom, a nitro group, a chlorine atom, a methoxy group, a methyl group or a phenyl group.
   [3] The anorectic of the above-mentioned [1], wherein the compound having a DGAT inhibitory activity is a compound represented by the following formula (2): 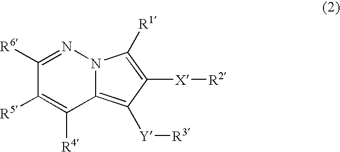
   wherein
X′ and Y′
   are the same or different and each is a single bond, a C₁₋₄ alkylene group, a C₂₋₄ heteroalkylene group, —O—, —CO₂—, —S(O)ₖ—,
   —C(O)—, —NR⁷′—, —C(O)NR⁷′—, —N(R⁸′)C(O)NR⁷′—, —N(R⁷′) CO₂—, —SO₂NR⁷′—, —N(R⁸′) SO₂NR⁷′—, —NR⁷′C(O)—, —O—C(O)N(R⁷′)- or —NR⁷′ SO₂—,
   wherein R⁷′ and R⁸′ are the same or different and each is a hydrogen atom, a C₁₋₈ alkyl group, an aryl group or an aryl C₁₋₄ alkyl group and k is an integer of 0 or 1-2;
R¹′ is a hydrogen atom, a halogen atom, a C₁₋₈ alkyl group, a C₂₋₈ alkenyl group, a C₂₋₈ alkynyl group, a C₁₋₈ fluoroalkyl group, a C₃₋₈ cycloalkyl group, a C₂₋₈ heteroalkyl group, a C₂₋₈ heteroalkenyl group; a C₃₋₈ heterocycloalkyl group, an aryl group, an aryl C₁₋₄ alkyl group, a heteroaryl group, OR^{a}′, SR^{a}′, C(O)R^{a}′, CO₂R^{a}′, C(O)NR^{a}′R^{b}′, SO₂R^{a}′, SO₂NR^{a}′R^{b}′ a nitro group or a cyano group, wherein R^{a}′ and R^{b}′ are the same or different and each is a hydrogen atom, a C₁₋₈ alkyl group, a C₃₋₈ cycloalkyl group, an aryl group or an aryl C₁₋₄ alkyl group;
R²′ is a C₁₋₈ alkyl group, an aryl C₁₋₄ alkyl group, OR^{a}′, a halogen atom, a nitro group, NR^{a}′R^{b}′, a cyano group or W¹′, wherein W¹′ is 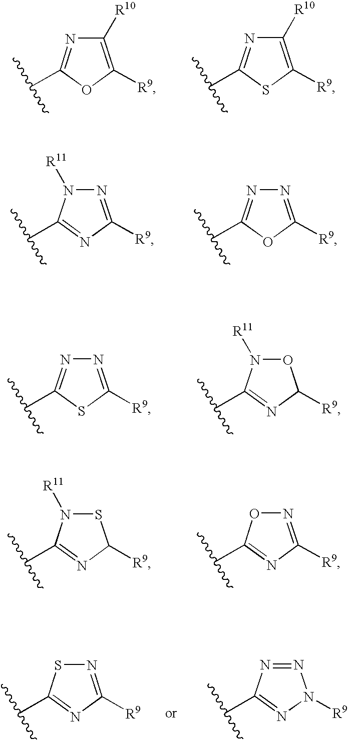
   wherein R⁹ and R¹⁰ are the same or different and each is a hydrogen atom, a C₁₋₈ alkyl group, a C₂₋₈ alkenyl group, a C₂₋₈ alkynyl group, a C₁₋₈ fluoroalkyl group, an aryl group or an aryl C₁₋₄ alkyl group, or R⁹ and R¹⁰ may be linked to form a 5 to 7-membered ring optionally having, in the ring, 1 to 3 heteroatoms selected from a nitrogen atom, an oxygen atom and a sulfur atom, R¹¹ is a hydrogen atom, a C₁₋₈ alkyl group, an aryl group or an aryl C₁₋₄ alkyl group, and R^{a}′ and R^{b}′ are as defined above; or
R¹′ and R²′
   may be linked to form a 5 to 7-membered ring optionally having, in the ring, one heteroatom selected from a nitrogen atom, an oxygen atom and a sulfur atom;
R³′ is a hydrogen atom, a C₁₋₈ alkyl group, an aryl C¹⁻⁴ alkyl group, OR^{a}′, a halogen atom, a nitro group, NR^{a}′R^{b}′, a cyano group or W²′, wherein W²′ is 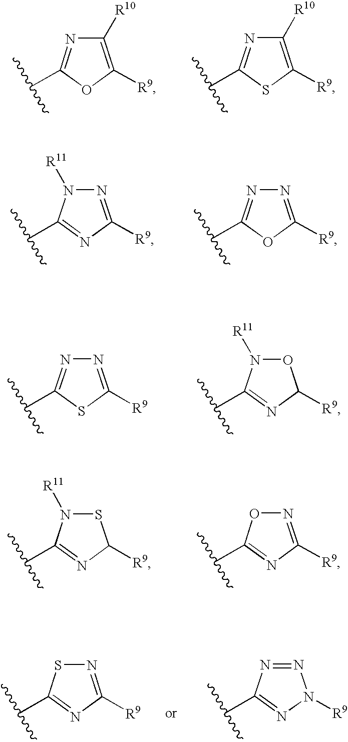
   wherein R⁹, R¹⁰ and R¹¹ are as defined above, and R^{a}′ and R^{b}′ are as defined above; or
R²′ and R³′
   may be linked to form a 5 to 7-membered ring optionally having, in the ring, one heteroatom selected from a nitrogen atom, an oxygen atom and a sulfur atom;
R⁴′ is a C₁₋₈ alkyl group, a C₂₋₈ alkenyl group, a C₂₋₈ alkynyl group, a C₁₋₄ fluoroalkyl group, a C₂₋₈ heteroalkyl group, a C₂₋₈ heteroalkenyl group, a C₃₋₈ cycloalkyl group, a C₃₋₈ heterocycloalkyl group, an aryl group, an aryl C₁₋₄ alkyl group, a heteroaryl group, OR^{a}′, SR^{a}′, NR^{a}′R^{b}′, C(O)R^{a}′, CO₂R^{a}′, C(O)NR^{a}′R^{b}′, SO₂R^{a} or SO₂NR^{a}′R^{b}′, wherein R^{a}′and R^{b′} are as defined above;
R⁵′, is a hydrogen atom, a C₁₋₈ alkyl group, a C₁₋₈ fluoroalkyl group, a C₃₋₈ cycloalkyl group, a C₂₋₈ heteroalkyl group, a C₂₋₈ heteroalkenyl group, a C₃₋₈ heterocycloalkyl group, an aryl group, an aryl C₁₋₄ alkyl group, a heteroaryl group, a halogen atom, OR^{a}′, NR^{a}′R^{b}′, a cyano group, C(O)R^{a}′, CO₂R^{a}′, C(O)NR^{a}′R^{b}′, OC(O)R^{a}′, OCO₂R^{c}, OC(O)NR^{a}′R^{b}′, NR^{a}′ C(O)R^{b}′, NR^{a}′CO₂R^{c} or NR^{a}′ C(O)NR^{a}′R^{b}′, wherein R^{a}′ and R^{b}′, are as defined above and R^{c} is a C₁₋₈ alkyl group, a C₃₋₈ cycloalkyl group, an aryl group or an aryl C₁₋₄ alkyl group; and
R⁶′ is OR^{d}, NR^{d}R^{e} or S(O)_{m′}R^{d},
   wherein R^{d} and R^{e} are the same or different and each is a hydrogen atom, a C₁₋₈ alkyl group, a C₂₋₈ alkenyl group, a C₂₋₈ alkynyl group, a C₁₋₈ fluoroalkyl group, C(O)R^{f}, an aryl group or an aryl C₁₋₄ alkyl group, m′ is an integer of 0 or 1-2, wherein R^{f} is a hydrogen atom, a C₁₋₈ alkyl group, an amino group, a C₁₋₄ alkylamino group, a di(C₁₋₄ alkyl)amino group, an aryl C₁₋₄ alkyl group or a C₁₋₈ alkoxy group, or when R⁶′ is NR^{d}R^{e}, R^{d} and R^{e} may form, together with the nitrogen atom binding thereto, an N-containing 4 to 7-membered heterocyclic ring wherein the ring may further contain 1 or 2 heteroatoms selected from a nitrogen atom, an oxygen atom and a sulfur atom.
      [4] The anorectic of the above-mentioned [1], wherein the compound having a DGAT inhibitory activity is a compound represented by the following formula (3): 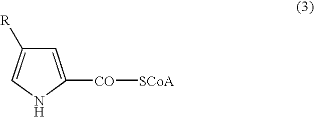
      wherein R is a C₅₋₂₅ alkyl group or a C₅₋₂₅ alkenyl group, and SCoA shows a residue which is a coenzyme A deficient in the hydrogen atom of a terminal mercapto group.
      [5] The anorectic of the above-mentioned [1], wherein the compound having a DGAT inhibitory activity is a compound represented by the following formula (4): 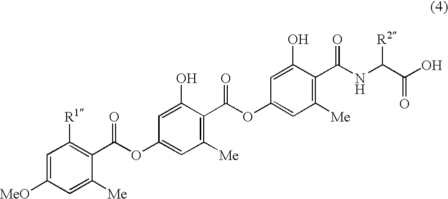
      wherein, when R¹″ is a hydrogen atom, R²″ is a methyl group or an isopropyl group, and when R¹″ is a methyl group, R²″ is a methyl group.
      [6] The anorectic of the above-mentioned [1], wherein the compound having a DGAT inhibitory activity is a compound represented by the following formula (5): 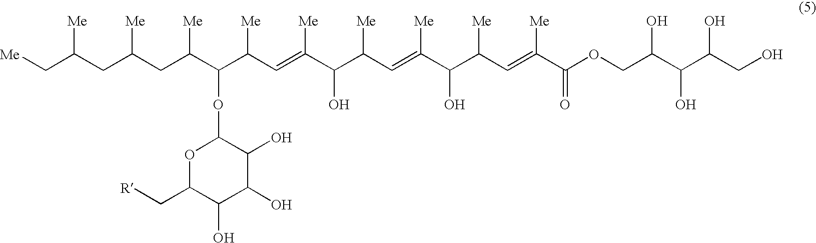
      wherein R′ is a hydroxyl group or an acetyloxy group.
      [7] The anorectic of the above-mentioned [1], wherein the compound having a DGAT inhibitory activity is a compound represented by the following formula (6): 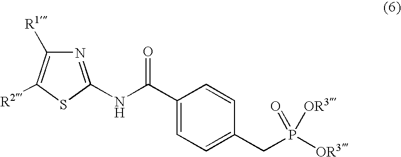
      wherein R′″ is a phenyl group or a halogen-substituted phenyl group, R²′″ is a hydrogen atom, a C₁₋₆ alkyl group, a carboxyl group, a C₁₋₆ alkoxy-carbonyl group, a cyano group, a C₁₋₆ alkyl-carbamoyl group, a N,N-di(C₁₋₆ alkyl)-carbamoyl group or a pyrrolidinocarbonyl group, and R³′″ is a C₁₋₆ alkyl group.
      [8] A method for suppressing appetite, which comprises administering a pharmaceutically effective amount of an anorectic of any of the above-mentioned [1] to [7] to a mammal.
      [9] A method for treating or preventing obesity, which comprises administering a pharmaceutically effective amount of an anorectic of any of the above-mentioned [1] to [7] to a mammal.
      [10] A method for treating or preventing hyperlipidemia, which comprises administering a pharmaceutically effective amount of an anorectic of any of the above-mentioned [1] to [7] to a mammal.
      [11] A method for treating or preventing diabetes, which comprises administering a pharmaceutically effective amount of an anorectic of any of the above-mentioned [1] to [7] to a mammal.
      [12] A method for treating or preventing arteriosclerosis, which comprises administering a pharmaceutically effective amount of an anorectic of any of the above-mentioned [1] to [7] to a mammal.
      [13] A method for treating or preventing a coronary disease, which comprises administering a pharmaceutically effective amount of an anorectic of any of the above-mentioned [1] to [7] to a mammal.
      [14] A method for treating or preventing hypertension, which comprises administering a pharmaceutically effective amount of an anorectic of any of the above-mentioned [1] to [7] to a mammal.
      [15] The method of the above-mentioned [9], which further comprises administering a pharmaceutically effective amount of other therapeutic agent for obesity to a mammal.
      [16] The method of the above-mentioned [15], wherein said other therapeutic agent for obesity is one or more drugs selected from the group consisting of mazindol, orlistat and sibutramine.
      [17] The method of the above-mentioned [10], which further comprises administering a pharmaceutically effective amount of other therapeutic agent for hyperlipidemia to a mammal.
      [18] The method of the above-mentioned [17], wherein said other therapeutic agent for hyperlipidemia is a statin drug.
      [19] The method of the above-mentioned [18], wherein the statin drug is one or more drugs selected from the group consisting of lovastatin, simvastatin, pravastatin, fluvastatin, atorvastatin, cerivastatin, pitavastatin, nisvastatin and rosuvastatin.
      [20] The method of the above-mentioned [17], wherein said other therapeutic agent for hyperlipidemia is a fibrate drug.
      [21] The method of the above-mentioned [20], wherein the fibrate drug is one or more drugs selected from the group consisting of clofibrate, clinofibrate, sinfibrate, fenofibrate, bezafibrate and gemfibrozil.
      [22] The method of the above-mentioned [17], wherein said other therapeutic agent for hyperlipidemia is probucol.
      [23] The method of the above-mentioned [17], wherein said other therapeutic agent for hyperlipidemia is nicotinic acid.
      [24] The method of the above-mentioned [17], wherein said other therapeutic agent for hyperlipidemia is a cholesterol absorption suppressant.
      [25] The method of the above-mentioned [24], wherein the cholesterol absorption suppressant is one or more drugs selected from the group consisting of ezetimibe, colestimide, colestyramine and colestipol.
      [26] The method of the above-mentioned [17], wherein said other therapeutic agent for hyperlipidemia is one or more drugs selected from the group consisting of an MTP inhibitor, an ACAT inhibitor and a CETP inhibitor.
      [27] The method of the above-mentioned [11], which further comprises administering a pharmaceutically effective amount of other therapeutic agent for diabetes to a mammal.
      [28] The method of the above-mentioned [27], wherein said other therapeutic agent for diabetes is one or more drugs selected from the group consisting of an insulin preparation, a sulfonylurea, an insulin secretagogue, a sulfonamide, a biguanide, an α glucosidase inhibitor and an insulin sensitizer.
      [29] The method of the above-mentioned [27], wherein said other therapeutic agent for diabetes is one or more drugs selected from the group consisting of insulin, glibenclamide, tolbutamide, glyclopyramide, acetohexamide, glimepiride, tolazamide, gliclazide, nateglinide, glybuzole, metformin hydrochloride, buformin hydrochloride, voglibose, acarbose and pioglitazone hydrochloride.
      [30] The method of the above-mentioned [12], which further comprises administering a pharmaceutically effective amount of other therapeutic agent for arteriosclerosis to a mammal.
      [31] The method of the above-mentioned [13], which further comprises administering a pharmaceutically effective amount of other therapeutic agent for coronary diseases to a mammal.
      [32] The method of the above-mentioned [14], which further comprises administering a pharmaceutically effective amount of other therapeutic agent for hypertension to a mammal.
      [33] The method of the above-mentioned [32], wherein said other therapeutic agent for hypertension is one or more drugs selected from the group consisting of a loop diuretic, an angiotensin converting enzyme inhibitor, an angiotensin II receptor antagonist, a Ca antagonist, a β blocker, an α,β blocker and an α blocker.
      [34] The method of the above-mentioned [32], wherein said other therapeutic agent for hypertension is one or more drugs selected from the group consisting of a furosemide sustained-release preparation, captopril, a captopril sustained-release preparation, enalapril maleate, alacepril, delapril hydrochloride, cilazapril, lisinopril, banazepril hydrochloride, imidapril hydrochloride, temocapril hydrochloride, quinapril hydrochloride, trandrapril, perindopril erbumine, losartan potassium, candesartan cilexetil, nicardipine hydrochloride, a nicardipine hydrochloride sustained-release preparation, nilvadipine, nifedipine, a nifedipine sustained-release preparation, benidipine hydrochloride, diltiazem hydrochloride, a diltiazem hydrochloride sustained-release preparation, nisoldipine, nitrendipine, manidipine hydrochloride, barnidipine hydrochloride, efonidipine hydrochloride, amlodipine besylate, felodipine, cilnidipine, aranidipine, propranolol hydrochloride, a propranolol hydrochloride sustained-release preparation, pindolol, a pindolol sustained-release preparation, indenolol hydrochloride, carteolol hydrochloride, a carteolol hydrochloride sustained-release preparation, bunitrolol hydrochloride, a bunitrolol hydrochloride sustained-release preparation, atenolol, acebutolol hydrochloride, metoprolol tartrate, a metoprolol tartrate sustained-release preparation, nipradilol, penbutolol sulfate, tilisolol hydrochloride, carvedilol, bisoprolol fumarate, betaxolol hydrochloride, celiprolol hydrochloride, bopindolol malonate, bevantolol hydrochloride, labetalol hydrochloride, arotinolol hydrochloride, amosulalol hydrochloride, prazosin hydrochloride, terazosin hydrochloride, doxazosin mesylate, bunazosin hydrochloride, a bunazosin hydrochloride sustained-release preparation, urapidil and phentolamine mesylate.

As is clear from the following test of the present invention, a compound having a DGAT inhibitory activity (e.g., DGAT1 inhibitory activity), a prodrug thereof and a pharmaceutically acceptable salt thereof showed a potent anorectic action. Accordingly, a compound having a DGAT inhibitory activity (e.g., DGAT1 inhibitory activity) is useful as an anorectic. Besides as the anorectic, it is also useful as an agent for treating or preventing diseases such as obesity, hyperlipidemia, diabetes, arteriosclerosis, coronary disease and hypertension.

Moreover, a compound having a DGAT inhibitory activity (e.g., DGAT1 inhibitory activity) is useful for combination therapy with other therapeutic agents for obesity, therapeutic agents for arteriosclerosis, therapeutic agents for coronary diseases, therapeutic agents for hypertension, therapeutic agents for diabetes or therapeutic agents for hyperlipidemia.

### BEST MODE FOR CARRYING OUT THE INVENTION

The definition of each substituent to be used in the present specification is as follows.

The “DGAT” refers to acyl CoA: diacylglycerol acyltransferase or a variant thereof. The diacylglycerol acyltransferase variants include proteins substantially homologous to native diacylglycerol acyltransferase. For example, proteins having one or more naturally or artificially occurring deletions, insertions or substitutions of amino acids, such as diacylglycerol acyltransferase derivatives, homologs and fragments can be mentioned. The amino acid sequence of a diacylglycerol acyltransferase variant is preferably at least about 80% identical, more preferably at least about 90% identical, and most preferably at least about 95% identical, to a native diacylglycerol acyltransferase.

The “halogen atom” is a chlorine atom, a bromine atom, a fluorine atom or an iodine atom.

The “C₁₋₈ alkyl group” is a straight or branched chain alkyl group having 1 to 8 (preferably 1 to 6) carbon atoms, and is exemplified by methyl group, ethyl group, n-propyl group, isopropyl group, n-butyl group, isobutyl group, sec-butyl group, tert-butyl group, n-pentyl group, isopentyl group, neopentyl group, tert-pentyl group, n-hexyl group, n-heptyl group, n-octyl group and the like. Of these, “C₁₋₆ alkyl group” refers to ones having 1 to 6 carbon atoms and “C₁₋₄ alkyl group” refers to ones having 1 to 4 carbon atoms.

The “C₅₋₂₅ alkyl group” is a straight or branched chain alkyl group having 5-25 (preferably 12-14) carbon atoms and is exemplified by decyl group, undecyl group, 2,2-dimethylundecyl group, 11,11′-dimethyldodecyl group, dodecyl group, 12-methyltridecyl group, tridecyl group, 12,12-dimethyltridecyl group, tetradecyl group, 6,6-dimethyltetradecyl group, pentadecyl group, hexadecyl group and the like.

The “C₁₋₈ fluoroalkyl group” is a straight or branched chain alkyl group having 1-8 (preferably 1-6) carbon atoms, which is substituted by 1-17 (preferably 1-5) fluorine atoms and is exemplified by fluoromethyl, difluoromethyl, trifluoromethyl, 1- or 2-fluoroethyl, 1,1-difluoroethyl, 1,2-difluoroethyl, 1-, 2- or 3-fluoropropyl, 1-, 2-, 3- or 4-fluorobutyl, 1-, 2-, 3-, 4- or 5-fluoropentyl, 1-, 2-, 3-, 4-, 5- or 6-fluorohexyl, 1-, 2-, 3-, 4-, 5-, 6- or 7-fluoroheptyl, 1-, 2-, 3-, 4-, 5-, 6-, 7- or 8-fluorooctyl and the like.

The “C₁₋₄ fluoroalkyl group” is a straight or branched chain alkyl group having 1 to 4 carbon atoms, which is substituted by 1-9 (preferably 1-5) fluorine atoms and is exemplified by fluoromethyl, difluoromethyl, trifluoromethyl, 1- or 2-fluoroethyl, 1,1-difluoroethyl, 1,2-difluoroethyl, 1-, 2- or 3-fluoropropyl, 1-, 2-, 3- or 4-fluorobutyl and the like.

The “C₂₋₈ heteroalkyl group” is a straight or branched chain heteroalkyl group comprising 2 to 8 (preferably 2 to 6) carbon atoms and 1 to 3 (preferably 1 or 2) heteroatoms. As the heteroatom, oxygen atom, nitrogen atom, silicon atom and sulfur atom can be mentioned, wherein the nitrogen and sulfur atoms may be oxidized and the nitrogen atom may be quaternized. The oxygen atom, nitrogen atom and sulfur atom may be present at any position other than the terminal and bond position. The silicon atom may be present at any position including the terminal and bond position. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —Si(CH₃)₃, —CH₂—CH═N—OCH₃ and the like. Up to two heteroatoms may be present in succession, as shown in, for example, —CH₂—NH—OCH₃, —CH₂—O—Si(CH₃)₃ and the like.

The “C₂₋₈ heteroalkenyl group” is a straight or branched chain heteroalkenyl group comprising 2 to 8 (preferably 2 to 6) carbon atoms and 1 to 3 (preferably 1 or 2) heteroatoms. As the heteroatom, oxygen atom, nitrogen atom, silicon atom and sulfur atom can be mentioned, wherein the nitrogen and sulfur atoms may be oxidized and the nitrogen atom may be quaternized. The oxygen atom, nitrogen atom and sulfur atom may be present at any position other than the terminal and bond position. The silicon atom may be present at any position including the terminal and bond position. Examples include —CH═CH—O—CH₃, —CH═CH₂—N(CH₃)₂ and the like. Up to two heteroatoms may be present in succession.

The “C₃₋₈ heterocycloalkyl group” comprises 3-8 (preferably 3-6) carbon atoms and 1-3 (preferably 1-2) heteroatoms, which are bonded in a ring. As the heteroatom, oxygen atom, nitrogen atom, silicon atom and sulfur atom can be mentioned. Of these, nitrogen atom and sulfur atom may be oxidized and nitrogen atom may be quaternized. The oxygen atom, nitrogen atom and sulfur atom may be present at any position except the bond position and silicon atom may be present at any position including the bond position. Up to two heteroatoms may be present in succession. Concrete examples include pyrrolidinyl, imidazolidinyl, pyrazolidinyl, piperidinyl, piperazinyl, tetrahydrofuranyl, 1,3-dioxolanyl, morpholinyl and the like.

The “C₃₋₈ heterocycloalkylene group” comprises 3-8 (preferably 3-6) carbon atoms and 1-3 (preferably 1-2) heteroatoms, which are bonded in a ring. As the heteroatom, oxygen atom, nitrogen atom, silicon atom and sulfur atom can be mentioned. Of these, nitrogen atom and sulfur atom may be oxidized and nitrogen atom may be quaternized. The oxygen atom, nitrogen atom and sulfur atom may be present at any position except the bond position and silicon atom may be present at any position including the bond position. Up to two heteroatoms may be present in succession. Concrete examples include divalent groups derived from the ring such as pyrrolidine, imidazolidine, pyrazolidine, piperidine, piperazine, tetrahydrofuran, 1,3-dioxolane, morpholine and the like.

The “C₁₋₈ alkoxy group” is a straight or branched chain alkoxy group having 1-8 (preferably 1-6) carbon atoms and is exemplified by methoxy group, ethoxy group, propoxy group, isopropoxy group, butoxy group, tert-butoxy group, pentyloxy group, tert-pentyloxy group, hexyloxy group and the like. Of these, the “C₁₋₆ alkoxy group” refers to those having 1-6 carbon atoms.

The “C₁₋₄ alkylamino group” is an amino group mono-substituted by straight or branched chain alkyl group having 1 to 4 carbon atoms. Examples thereof include methylamino group, ethylamino group, propylamino group, butylamino group and the like.

The “di(C₁₋₄ alkyl)amino group” is an amino group di-substituted by straight or branched chain alkyl group having 1 to 4 carbon atoms, wherein the alkyl moieties may be the same or different. Examples thereof include dimethylamino group, diethylamino group, dipropylamino group, dibutylamino group and the like.

The “C₃₋₈ cycloalkyl group” is a cycloalkyl group having 3-8 (preferably 3-7) carbon atoms. Concrete examples include cyclopropyl group, cyclobutyl group, cyclopentyl group, cyclohexyl group, cycloheptyl group and cyclooctyl group and the like.

The “cycloalkyl group” is a cycloalkyl group preferably having 3-8 (more preferably 3-7) carbon atoms. Concrete examples include cyclopropyl group, cyclobutyl group, cyclopentyl group, cyclohexyl group, cycloheptyl group and cyclooctyl group and the like.

The “C₃₋₈ cycloalkylene group” is a cycloalkylene group having 3-8 (preferably 3-7) carbon atoms. Concrete examples include cyclopropylene group, cyclobutylene group, cyclopentylene group, cyclohexylene group, cycloheptylene group and cyclooctylene group and the like.

The “aryl group” is an aromatic hydrocarbon group preferably having 6-12, more preferably 6-10, carbon atoms and the number of the rings is 1-3 (preferably 1-2). When aryl group comprises a plurality of rings, they may be condensed with each other to form a fused ring or bonded via a covalent bond. Concrete examples include, but not limited to, phenyl group, 1-naphthyl group, 2-naphthyl group, 4-biphenylyl group, 1,2,3,4-tetrahydronaphthyl group and the like.

The “arylene group” is a divalent aromatic hydrocarbon group preferably having 6-12, more preferably 6-10, carbon atoms and the number of the rings is 1-3 (preferably 1-2). When the arylene group comprises a plurality of rings, they may be condensed with each other to form a fused ring or bonded via a covalent bond. Concrete examples include, but not limited to, phenylene group, naphthylene group, biphenylene group, 1,2,3,4-tetrahydronaphthylene group and the like.

The “heteroaryl group” is a heteroaryl group having at least 1 (preferably 1-4) heteroatoms selected from nitrogen atom, oxygen atom and sulfur atom. Of the heteroatoms, nitrogen atom and sulfur atom may be oxidized and nitrogen atom may be quaternized. The heteroaryl group is preferably a 5 or 6-membered ring. The heteroaryl group may comprise a plurality of rings and, in that case, they may be condensed with each other to form a fused ring. The heteroaryl group includes a fused ring with a benzene ring. Concrete examples include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 5-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, 6-quinolyl and the like. The heteroaryl group may be substituted by phenyl group (e.g., 2-phenyl-4-oxazolyl and the like).

The “heteroarylene group” is a heteroarylene group having at least 1 (preferably 1-4) heteroatom selected from nitrogen atom, sulfur atom and oxygen atom. Of the heteroatoms, nitrogen atom and sulfur atom may be oxidized and nitrogen atom may be quaternized. The heteroarylene group is preferably a 5 or 6-membered ring. The heteroarylene group may comprise a plurality of rings and, in that case, they may be condensed with each other to form a fused ring. The heteroarylene group includes a fused ring with a benzene ring. Concrete examples include divalent groups derived from the ring such as pyrrole, pyrazole, imidazole, pyrazine, oxazole, isoxazole, thiazole, furan, thiophene, pyridine, pyrimidine, benzothiazole, purine, benzimidazole, indole, isoquinoline, quinoxaline, quinoline and the like. The heteroarylene group may be substituted by phenyl group (e.g., a divalent group derived from 2-phenyl-4-oxazole and the like).

The “aryl C₁₋₄ alkyl group” is a group wherein an aryl group is bonded to an alkyl group, wherein the aryl moiety includes both scopes of the above-mentioned “aryl group” and “heteroaryl group” and the alkyl moiety is a straight or branched chain alkyl group having 1-4 (preferably 1-3) carbon atoms. It also encompasses a group in which the carbon atom of the alkyl moiety is substituted by, for example, oxygen atom. Concrete examples include benzyl, phenethyl, pyridylmethyl, phenoxymethyl, 2-pyridyloxymethyl, 3-(1-naphthyloxy)propyl and the like.

The “C₁₋₄ alkylene group” and the C₁₋₄ alkylene moiety of “C₁₋₄ alkylene-OR^{a} group” is a straight or branched chain alkylene group having 1 to 4 carbon atoms. Specific examples include —CH₂—, —CH₂—CH₂—, —CH₂—CH₂—CH₂—, —CH₂—CH₂—CH₂—CH₂—, —CH(CH₃)—, —CH(CH₃)—CH₂—, —CH(CH₃)—CH₂—CH₂—, —CH₂—CH(CH₃)—CH₂— and the like.

The “C₂₋₄ heteroalkylene group” is a straight or branched chain heteroalkylene group comprising 2-4 carbon atoms and at least 1 (preferably 1-2) heteroatom. As the heteroatom, nitrogen atom, oxygen atom and sulfur atom can be mentioned, which may be positioned at a terminal which may be one or both of the terminals. Specific examples include —CH₂—CH₂—S—CH₂—CH₂—, —O—CH₂—CH₂—CH₂—CH₂—, —O—CH₂—CH₂—CH₂—CH₂—O—, —NH—CH₂—CH₂—CH₂—CH₂—, —O—CH₂—CH₂—CH₂—CH₂—NH—, —CH₂ (CH₃)—S—CH₂—, —O—CH₂ (CH₃)—CH₂—CH₂—, —O—CH₂ (CH₃)—CH₂—CH₂—O—, —NH—CH₂ (CH₃)—CH₂—CH₂—, —O—CH₂ (CH₃)—CH₂—CH₂—NH— and the like.

The “C₁₋₄ heteroalkylene group” is a straight or branched chain heteroalkylene group comprising 1-4 carbon atoms and at least 1 (preferably 1-2) heteroatom. As the heteroatom, nitrogen atom, oxygen atom and sulfur atom can be mentioned. They may be positioned at a terminal which may be one or both of the terminals. Concrete examples include —CH₂—CH₂—S—CH₂—CH₂—, —O—CH₂—CH₂—CH₂—CH₂—, —O—CH₂—CH₂—CH₂—CH₂—O—, —NH—CH₂—CH₂—CH₂—CH₂—, —O—CH₂—CH₂—CH₂—CH₂—NH—, —CH₂ (CH₃)—S—CH₂—, —O—CH₂ (CH₃)—CH₂—CH₂—, —O—CH₂ (CH₃)—CH₂—CH₂—O—, —NH—CH₂ (CH₃)—CH₂—CH₂—, —O—CH₂ (CH₃)—CH₂—CH₂—NH— and the like.

The “C₂₋₈ alkenyl group” is a straight or branched chain alkenyl having 2-8 (preferably 2-6) carbon atoms, which includes one or more double bonds. Examples include vinyl, 2-propenyl, allyl, crotyl, 2-isopentenyl, 1,3-butadien-2-yl, 2,4-pentadienyl, 1,4-pentadien-3-yl and the like, and isomers thereof.

The “C₅₋₂₅ alkenyl group” is a straight or branched chain alkenyl group having 5-25 (preferably 12-14) carbon atoms and is exemplified by 1-decenyl, 4,7-decadienyl, 10-methyl-9-undecenyl, 2-undecenyl, 4,8-dimethyl-3,7-nonadienyl, 1-dodecenyl, 2-tridecenyl, 6-tridecenyl, 1-tetradecenyl, 3,7,11-trimethyl-2,6,10-dodecatrienyl, 1-pentadecenyl, 1-hexadecenyl and the like.

The “C₂₋₈ alkynyl group” is a straight or branched chain alkynyl group having 2-8 (preferably 2-6) carbon atoms, which includes one or more triple bonds. Concrete examples include ethynyl, 1-propynyl, 3-propynyl, 3-butynyl and the like, and isomers thereof.

The “5 to 7-membered ring” is a carbocycle or heterocycle which is saturated or unsaturated and aromatic or aliphatic. The 5 to 7-membered ring formed by a substituent of W² and a substituent of W¹ in combination is condensed with W¹ to form a fused ring or spiro ring with W². As the heteroatom to constitute heterocycle, nitrogen atom, oxygen atom, sulfur atom and the like can be mentioned, and 1-3, preferably 1-2, of these are contained. Concrete examples include cycloalkane (e.g., cyclopentane, cyclohexane etc.), cycloalkene (e.g., cyclopentene, cyclohexene etc.), arene (e.g., benzene) and heterocycle (e.g., furan, thiophene, pyrrole, oxazole, isoxazole, thiazole, isothiazole, imidazole, pyrazole, pyridine, pyridazine, pyrimidine and a hydrogenated compound thereof etc.). Of these, the “5 or 6-membered ring” refers to those that are 5 or 6-membered rings.

The “5 to 7-membered ring optionally having, in the ring, 1 to 3 heteroatoms selected from a nitrogen atom, an oxygen atom and a sulfur atom” may be saturated or unsaturated and aromatic or aliphatic. Concrete examples include cycloalkane (e.g., cyclopentane, cyclohexane etc.), cycloalkene (e.g., cyclopentene, cyclohexene etc.), arene (e.g., benzene) and heterocycle (e.g., furan, thiophene, pyrrole, oxazole, isoxazole, thiazole, isothiazole, imidazole, pyrazole, pyridine, pyridazine, pyrimidine and a hydrogenated compound thereof etc.).

The “5 to 7-membered ring optionally having, in the ring, one heteroatom selected from a nitrogen atom, an oxygen atom and a sulfur atom” may be saturated or unsaturated and aromatic or aliphatic. Concrete examples include cycloalkane (e.g., cyclopentane, cyclohexane etc.), cycloalkene (e.g., cyclopentene, cyclohexene etc.), arene (e.g., benzene) and heterocycle (e.g., furan, thiophene, pyrrole, pyran, pyridine and a hydrogenated compound thereof etc.).

The “N-containing 5 to 7-membered ring” may be saturated or unsaturated and aromatic or aliphatic, and contains at least one nitrogen atom in the ring, and further may have a heteroatom selected from nitrogen atom, oxygen atom and sulfur atom. Concrete examples include pyrrole, oxazole, isoxazole, thiazole, isothiazole, imidazole, pyrazole, pyridine, pyridazine, pyrimidine, a hydrogenated compound thereof and the like.

The “C₂₋₄ alkenylene group” is a straight or branched chain alkenylene group having 2-4 carbon atoms. Concrete examples include 1-propen-1,3-diyl, 2-propen-1,3-diyl, 1-butene-1,4-diyl, 2-butene-1,4-diyl, 3-butene-1,4-diyl, 1,3-butadien-1,4-diyl and the like.

The “3 to 6-membered ring” is a carbocycle or heterocycle which is saturated or unsaturated and aromatic or aliphatic. As the heteroatom to constitute heterocycle, nitrogen atom, oxygen atom, sulfur atom and the like can be mentioned, and 1-3, preferably 1-2, of these are contained. Concrete examples include cycloalkane (e.g., cyclopropane, cyclobutane, cyclopentane, cyclohexane etc.), cycloalkene (e.g., cyclopropene, cyclobutene, cyclopentene, cyclohexene etc.), arene (e.g., benzene) and heterocycle (e.g., furan, thiophene, pyrrole, oxazole, isoxazole, thiazole, isothiazole, imidazole, pyrazole, pyridine, pyridazine, pyrimidine and a hydrogenated compound thereof etc.).

The “N-containing 4 to 7-membered heterocycle” is a saturated or unsaturated 4 to 7-membered heterocycle containing at least one nitrogen atom. The ring may further contain 1-2 heteroatoms selected from nitrogen atom, oxygen atom and sulfur atom. Concrete examples include pyrrole, oxazole, isoxazole, thiazole, isothiazole, imidazole, pyrazole, pyridine, pyridazine, pyrimidine, a hydrogenated compound thereof and the like.

The “halogen-substituted phenyl group” is a phenyl group substituted by 1-5 halogen atoms and is exemplified by 4-chlorophenyl, 4-bromophenyl, 2-chlorophenyl, 3-chlorophenyl, 3-bromophenyl, 2-fluorophenyl, 4-fluorophenyl, 4-iodophenyl, 2,3-dichlorophenyl, 3,4-dichlorophenyl, 2,4-dichlorophenyl, 2,4,6-trichlorophenyl and the like.

The “C₁₋₆ alkoxy-carbonyl group” is a carbonyl group substituted by the above-mentioned “C₁₋₆ alkoxy group” and is exemplified by methoxycarbonyl, ethoxycarbonyl, propoxycarbonyl, isopropoxycarbonyl, butoxycarbonyl, isobutoxycarbonyl, tert-butoxycarbonyl, pentyloxycarbonyl, hexyloxycarbonyl and the like.

The “C₁₋₆ alkyl-carbamoyl group” is a carbamoyl group mono-substituted by the above-mentioned “C₁₋₆ alkyl group” and is exemplified by methylcarbamoyl, ethylcarbamoyl, propylcarbamoyl, isopropylcarbamoyl, butylcarbamoyl, isobutylcarbamoyl, tert-butylcarbamoyl, pentylcarbamoyl, hexylcarbamoyl and the like.

The “N,N-di(C₁₋₆ alkyl)-carbamoyl group” is a carbamoyl group di-substituted by the above-mentioned “C₁₋₆ alkyl group” and is exemplified by N,N-dimethylcarbamoyl, N,N-diethylcarbamoyl, N,N-dipropylcarbamoyl, N,N-dibutylcarbamoyl, N,N-dipentylcarbamoyl, N,N-dihexylcarbamoyl and the like.

The “C₃₋₈ cycloalkylene group”, “C₃₋₈ heterocycloalkylene group”, “arylene group” and “heteroarylene group” for W¹ are optionally substituted by preferably 1 to 4, more preferably 1 or 2, particularly preferably one substituent mentioned below. As the substituent, halogen atom, R^{c1}, —OR^{c1}, —N(R^{c1})₂, SR^{c1}, cyano group, nitro group, C₁₋₈ alkyl group, C₂₋₈ alkenyl group, C₂₋₈ alkynyl group and the like can be specifically mentioned. As used herein, R^{c1} is a hydrogen atom, C₁₋₈ alkyl group, C₂₋₈ alkenyl group, C₂₋₈ alkynyl group and the like, and R^{c1}s for —N(R^{c1})₂ are the same or different and may be linked to form a 5 or 6-membered ring.

The “C₃₋₈ cycloalkyl group”, “C₃₋₈ heterocycloalkyl group”, “aryl group” and “heteroaryl group” for W² are optionally substituted by preferably 1 to 4, more preferably 1 or 2, substituents mentioned below. As the substituent, halogen atom, R^{d1}, —OR^{d1}, N(R^{d1})₂, —(CH₂) t—S(O)uR^{e1}, cyano group, nitro group, C₁₋₈ haloalkyl group, C₁₋₈ haloalkoxy group, aryl C₁₋₄ alkyl group, heteroaryl C₁₋₄ alkyl group, —CH(R^{f1})—CO₂R^{e1}, —C(R^{f1})₂—CO₂R^{e1}, —C(O)CO₂R^{e1}, ═CH—CONR^{e1}R^{f1}, ═CH—CO₂R^{e1}, —(CH₂)t—CO₂R^{e1}, —(CH₂)t—C(O)R^{e1}, —(CH₂)t—C(O)NR^{e1}R^{f1}, —(CH₂)t—NHSO₂R^{e1}, —(CH₂)t—NHSO₂NR^{e1}R^{f1}, —(CH₂)t—NR^{e1}R^{f1}, —(CH₂)t—OR^{e1}, —(CH₂)t—NHSO₂NHCO₂R^{e1}, —(CH₂)t—NHSO₂NR^{e1}R^{f1}, —(CH₂)t—CONHSO₂R^{e1}, —(CH₂)t—W³, —(CH₂)t—NHCO₂R^{e1}, —(CH₂)t—NR^{f1}COR^{e1}, (CH₂)t—NHCONR^{e1}R^{f1}, —(CH₂)t—NHCO—(CH₂)t—OCOR^{e1}, wherein t is an integer of 0-8, u is an integer of 0-2, R^{d1} is hydrogen atom, C₁₋₈ alkyl group, C₂₋₈ alkenyl group, C₂₋₈ alkynyl group or C₃₋₈ cycloalkyl group, wherein the aliphatic moiety is optionally substituted by hydroxyl group, carboxyl group, amino group, carbamoyl group, phenyl group, halogen atom, C₁₋₄ haloalkyl group or —CO₂R^{g} (wherein R^{g} is C₁₋₄ alkyl group), two R^{d1}s may form, together with the adjacent nitrogen atom, a 5 or 6-membered ring, R^{e1 and R}^{f1} are the same or different and each is hydrogen atom or C₁₋₈ alkyl group, or may form a 5 or 6-membered ring together with the adjacent nitrogen atom, the alkyl moieties of R^{e1} and R^{f1} may be substituted by hydroxyl group, carboxyl group, amino group, carbamoyl group, phenyl group, dialkylamino group or —CO₂R^{g} (wherein R^{g} is as defined above), and W³ is an optionally substituted aryl group, optionally substituted aralkyl group, optionally substituted heterocyclic group or optionally substituted cycloalkyl group, and the like can be specifically mentioned.

The “C₁₋₈ haloalkyl group” is a straight or branched chain alkyl group having 1-8 (preferably 1-6) carbon atoms, which is substituted by 1-17 (preferably 1-5) halogen atoms, and is exemplified by fluoromethyl, difluoromethyl, trifluoromethyl, 1- or 2-fluoroethyl, 1,1-difluoroethyl, 1,2-difluoroethyl, 1-, 2- or 3-fluoropropyl, 1-, 2-, 3- or 4-fluorobutyl, 1-, 2-, 3-, 4- or 5-fluoropentyl, 1-, 2-, 3-, 4-, 5- or 6-fluorohexyl, 1-, 2-, 3-, 4-, 5-, 6- or 7-fluoroheptyl, 1-, 2-, 3-, 4-, 5-, 6-, 7- or 8-fluorooctyl and the like; bromomethyl, dibromomethyl, tribromomethyl, 1- or 2-bromoethyl, 1,1-dibromoethyl, 1,2-dibromoethyl, 1-, 2- or 3-bromopropyl, 1-, 2-, 3- or 4-bromobutyl, 1-, 2-, 3-, 4- or 5-bromopentyl, 1-, 2-, 3-, 4-, 5- or 6-bromohexyl, 1-, 2-, 3-, 4-, 5-, 6- or 7-bromoheptyl, 1-, 2-, 3-, 4-, 5-, 6-, 7- or 8-bromooctyl and the like; chloromethyl, dichloromethyl, trichloromethyl, 1- or 2-chloroethyl, 1,1-dichloroethyl, 1,2-dichloroethyl, 1-, 2- or 3-chloropropyl, 1-, 2-, 3- or 4-chlorobutyl, 1-, 2-, 3-, 4- or 5-chloropentyl, 1-, 2-, 3-, 4-, 5- or 6-chlorohexyl, 1-, 2-, 3-, 4-, 5-, 6- or 7-chloroheptyl, 1-, 2-, 3-, 4-, 5-, 6-, 7- or 8-chlorooctyl and the like; iodomethyl, diiodomethyl, triiodomethyl, 1- or 2-iodoethyl, 1,1-diiodoethyl, 1,2-diiodoethyl, 1-, 2- or 3-iodopropyl, 1-, 2-, 3- or 4-iodobutyl, 1-, 2-, 3-, 4- or 5-iodopentyl, 1-, 2-, 3-, 4-, 5- or 6-iodohexyl, 1-, 2-, 3-, 4-, 5-, 6- or 7-iodoheptyl, 1-, 2-, 3-, 4-, 5-, 6-, 7- or 8-iodooctyl and the like. Of these, the “C₁₋₄ haloalkyl group” refers to those having 1-4 carbon atoms.

The “C₁₋₈ haloalkoxy group” is a straight or branched chain alkoxy group having 1-8 (preferably 1-6) carbon atoms, which is substituted by 1-17 (preferably 1-5) halogen atoms, and is exemplified by fluoromethoxy, difluoromethoxy, trifluoromethoxy, 1- or 2-fluoroethoxy, 1,1-difluoroethoxy, 1,2-difluoroethoxy, 1-, 2- or 3-fluoropropoxy, 1-, 2-, 3- or 4-fluorobutoxy, 1-, 2-, 3-, 4- or 5-fluoropentyloxy, 1-, 2-, 3-, 4-, 5- or 6-fluorohexyloxy, 1-, 2-, 3-, 4-, 5-, 6- or 7-fluoroheptyloxy, 1-, 2-, 3-, 4-, 5-, 6-, 7- or 8-fluorooctyloxy and the like; bromomethoxy, dibromomethoxy, tribromomethoxy, 1- or 2-bromoethoxy, 1,1-dibromoethoxy, 1,2-dibromoethoxy, 1-, 2- or 3-bromopropoxy, 1-, 2-, 3- or 4-bromobutoxy, 1-, 2-, 3-, 4- or 5-bromopentyloxy, 1-, 2-, 3-, 4-, 5- or 6-bromohexyloxy, 1-, 2-, 3-, 4-, 5-, 6- or 7-bromoheptyloxy, 1-, 2-, 3-, 4-, 5-, 6-, 7- or 8-bromooctyloxy and the like; chloromethoxy, dichloromethoxy, trichloromethoxy, 1- or 2-chloroethoxy, 1,1-dichloroethoxy, 1,2-dichloroethoxy, 1-, 2- or 3-chloropropoxy, 1-, 2-, 3- or 4-chlorobutoxy, 1-, 2-, 3-, 4- or 5-chloropentyloxy, 1-, 2-, 3-, 4-, 5- or 6-chlorohexyloxy, 1-, 2-, 3-, 4-, 5-, 6- or 7-chloroheptyloxy, 1-, 2-, 3-, 4-, 5-, 6-, 7- or 8-chlorooctyloxy and the like; iodomethoxy, diiodomethoxy, triiodomethoxy, 1- or 2-iodoethoxy, 1,1-diiodoethoxy, 1,2-diiodoethoxy, 1-, 2- or 3-iodopropoxy, 1-, 2-, 3- or 4-iodobutoxy, 1-, 2-, 3-, 4- or 5-iodopentyloxy, 1-, 2-, 3-, 4-, 5- or 6-iodohexyloxy, 1-, 2-, 3-, 4-, 5-, 6- or 7-iodoheptyloxy, 1-, 2-, 3-, 4-, 5-, 6-, 7- or 8-iodooctyloxy and the like.

The “heteroaryl C₁₋₄ alkyl group” is a straight or branched chain alkyl having 1 to 4 carbon atoms, which is substituted by the above-mentioned “heteroaryl”. Concrete examples include pyrrolylmethyl, imidazolylmethyl, oxazolylmethyl, isoxazolylmethyl, thiazolylmethyl, furylmethyl, thienylmethyl, pyridylmethyl, pyrimidylmethyl, indolylmethyl, isoquinolylmethyl, tetrazolylmethyl, oxadiazolylmethyl, piperidinylmethyl, pyrrolylethyl, imidazolylethyl, oxazolylethyl, isoxazolylethyl, thiazolylethyl, furylethyl, thienylethyl, pyridylethyl, pyrimidylethyl, indolylethyl, isoquinolylethyl, tetrazolylethyl, oxadiazolylethyl, piperidinylethyl, pyrrolylpropyl, imidazolylpropyl, oxazolylpropyl, isoxazolylpropyl, thiazolylpropyl, furylpropyl, thienylpropyl, pyridylpropyl, pyrimidylpropyl, indolylpropyl, isoquinolylpropyl, tetrazolylpropyl, oxadiazolylpropyl, piperidinylpropyl, pyrrolylbutyl, imidazolylbutyl, oxazolylbutyl, isoxazolylbutyl, thiazolylbutyl, furylbutyl, thienylbutyl, pyridylbutyl, pyrimidylbutyl, indolylbutyl, isoquinolylbutyl, tetrazolylbutyl, oxadiazolylbutyl, piperidinylbutyl and the like (including all isomers). The heteroaryl moiety may be substituted by oxo, C₁₋₄ alkyl group (e.g., methyl etc.), hydroxyl group and the like.

The “dialkylamino group” is an amino group di-substituted by alkyl group, wherein each alkyl preferably has 1-6, more preferably 1-4, carbon atoms, and is a straight or branched chain. The alkyl moieties may be the same or different. Examples thereof include dimethylamino, diethylamino, dipropylamino, dibutylamino and the like.

The “C₁₋₄ hydroxyalkyl group” is a straight or branched chain alkyl group having 1 to 4 carbon atoms, which is substituted by hydroxy group. Examples thereof include hydroxymethyl, 1- or 2-hydroxyethyl, 1-, 2- or 3-hydroxypropyl, 1-, 2-, 3- or 4-hydroxybutyl and the like.

The “C₁₋₆ alkoxy C₁₋₄ alkyl group” is the above-mentioned “C₁₋₄ alkyl group” substituted by the above-mentioned “C₁₋₆ alkoxy”. Concrete examples include methoxymethyl, ethoxymethyl, propoxymethyl, isopropoxymethyl, butoxymethyl, 1- or 2-methoxyethyl, 1- or 2-ethoxyethyl, 1- or 2-propoxyethyl, 1- or 2-isopropoxyethyl, 1- or 2-butoxyethyl, 1-, 2- or 3-methoxypropyl, 1-, 2- or 3-ethoxypropyl, 1-, 2- or 3-propoxypropyl, 1-, 2- or 3-isopropoxypropyl, 1-, 2- or 3-butoxypropyl, 1-, 2-, 3- or 4-methoxybutyl, 1-, 2-, 3- or 4-ethoxybutyl, 1-, 2-, 3- or 4-propoxybutyl, 1-, 2-, 3- or 4-isopropoxybutyl, 1-, 2-, 3- or 4-butoxybutyl and the like.

The C₁₋₄ alkyl moiety of the “—COO—C₁₋₄ alkyl group” is as defined above for the “C₁₋₄ alkyl group”. Concrete examples include methoxycarbonyl, ethoxycarbonyl, propoxycarbonyl, isopropoxycarbonyl, butoxycarbonyl and the like.

The “aryl group” of W³ is optionally substituted and as the substituent, the groups that substitute the “aryl group” for W² can be mentioned.

The “aralkyl group” is a group in which straight or branched chain alkyl group having preferably 1-6 (more preferably 1-4) carbon atoms is bonded to aryl group (as defined above). Concrete examples include benzyl, 1- or 2-phenylethyl, 1-, 2- or 3-phenylpropyl, 1-, 2-, 3- or 4-phenylbutyl, naphthylmethyl and the like.

The “heterocyclic group” is a saturated or unsaturated, preferably 4 to 8-membered, more preferably 5 to 7-membered, heterocycle. The heterocycle contains 1-3 heteroatoms selected from nitrogen atom, oxygen atom and sulfur atom. For example, furan, thiophene, pyrrole, oxazole, isoxazole, thiazole, isothiazole, imidazole, pyrazole, pyridine, pyridazine, pyrimidine and a hydrogenated compound thereof and the like can be mentioned.

The “aralkyl group”, “cycloalkyl group” and “heterocyclic group” for W³ are optionally substituted and, as the substituent, for example, C₁₋₈ alkyl group, hydroxyl group, nitro group, cyano group, C₁₋₈ alkoxy group, amino group, carboxyl group, C₁₋₈ haloalkyl group and the like can be mentioned.

The “pharmaceutically acceptable salt” includes, for example, salts with sodium, potassium, calcium, ammonia, organic amine, magnesium and the like, or similar salts, when a compound having a DGAT inhibitory activity (e.g., DGAT1 inhibitory activity) contains an acidic group. When the compound having a DGAT inhibitory activity (e.g., DGAT1 inhibitory activity) contains a basic group, for example, various inorganic acid addition salts such as hydrochloride, hydrobromide, carbonate, hydrogen carbonate, phosphate, monohydrogen phosphate, dihydrogen phosphate, sulfate, hydrogen sulfate, hydrochloride, nitrate and the like; and various organic acid addition salts such as acetate, propionate, isobutyrate, malonate, benzoate, suberate, mandelate, phthalate, tartrate, citrate, methanesulfonate, benzenesulfonate, p-toluenesulfonate and the like can be mentioned. Salts with various amino acids such as arginine and the like, and salts with organic acids such as glucuronic acid, galactunoric acid and the like are also included (Berge, S. M., et al, “Pharmaceutical Salts”, Journal of Pharmaceutical Science, 66, 1-19, 1977). Furthermore, when the compound contains both the basic and acidic groups, both the salts with acid and the salts with base are included. Water-containing product, hydrate and solvate may be also included.

When a compound having a DGAT inhibitory activity (e.g., DGAT1 inhibitory activity) contains various isomers, the compound also encompasses such isomers. For example, E form and Z form can be present as geometric isomers, and when an asymmetric carbon atom exists, enantiomer and diastereomer can be present as stereoisomers based thereon, and tautomer can be also present. Accordingly, the present invention encompasses all these isomers and mixtures thereof. In addition, the present invention also encompasses a prodrug and a metabolite thereof.

The “prodrug” in the present invention has a group capable of chemical or metabolic decomposition, and is a derivative of a compound having a DGAT inhibitory activity (e.g., DGAT1 inhibitory activity), which shows pharmaceutical activity by hydrolysis or solvolysis, or decomposition under physiological conditions. For example, compounds wherein a hydroxyl group thereof has been substituted by —CO-alkyl, —CO₂-alkyl, —CONH-alkyl, —CO-alkenyl, —CO₂-alkenyl, —CONH-alkenyl, —CO-aryl, —CO₂-aryl, —CONH-aryl, —CO-heterocycle, —CO₂-heterocycle, —CONH-heterocycle (the alkyl, alkenyl, aryl and heterocycle are optionally substituted by halogen atom, alkyl group, hydroxyl group, alkoxy group, carboxy group, amino group, amino acid residue, —PO₃H₂, —SO₃H, —OPO₃H₂, —OSO₃H etc.), —CO-polyethylene glycol residue, —CO₂-polyethylene glycol residue, —CO-polyethylene glycol monoalkyl ether residue, —CO₂-polyethylene glycol mono-alkyl ether residue, —PO₃H₂ and the like,

compounds wherein an amino group thereof has been substituted by —CO-alkyl, —CO₂-alkyl, —CO-alkenyl, —CO₂-alkenyl, —CO₂-aryl, —CO-aryl, —CO-heterocycle, —CO₂-heterocycle (the alkyl, alkenyl, aryl and heterocycle are optionally substituted by halogen atom, alkyl group, hydroxyl group, alkoxy group, carboxy group, amino group, amino acid residue, —PO₃H₂, —SO₃H, —OPO₃H₂, —OSO₃H etc.), —CO-polyethylene glycol residue, —CO₂-polyethylene glycol residue, —CO-polyethylene glycol mono-alkyl ether residue, —CO₂-polyethylene glycol mono-alkyl ether residue, —PO₃H₂ and the like, and

compounds wherein a carboxy group thereof has been substituted by alkoxy group, aryloxy group (the alkoxy group and aryloxy group are optionally substituted by halogen atom, alkyl group, hydroxyl group, alkoxy group, carboxy group, amino group, amino acid residue, —PO₃H₂, —SO₃H, —OPO₃H₂, —OSO₃H etc.), polyethylene glycol residue or polyethylene glycol monoalkyl ether residue and the like can be mentioned.

The present inventors have first found that a compound having a DGAT inhibitory activity (e.g., DGAT1 inhibitory activity) is useful as an anorectic. Therefore, a compound having a DGAT inhibitory activity (e.g., DGAT1 inhibitory activity) is considered to be also useful as an agent for treating or preventing obesity. Moreover, it is considered to be useful as an agent for treating or preventing hyperlipidemia, diabetes, arteriosclerosis, coronary disease or hypertension. The present inventors have also found that, when using as an agent for treating or preventing these diseases, concurrent use thereof with other pharmaceutical agents affords its effect.

As a compound having a DGAT inhibitory activity (e.g., DGAT1 inhibitory activity), compounds represented by the above-mentioned formulas (1)-(6) can be mentioned. The compound represented by the above-mentioned formula (1) can be produced by a synthetic technique known in the art using commercially available starting materials. The production method of the compound of the above-mentioned formula (1) wherein, for example, X is N, Y is N and Z is O, is shown in Scheme 1-1. 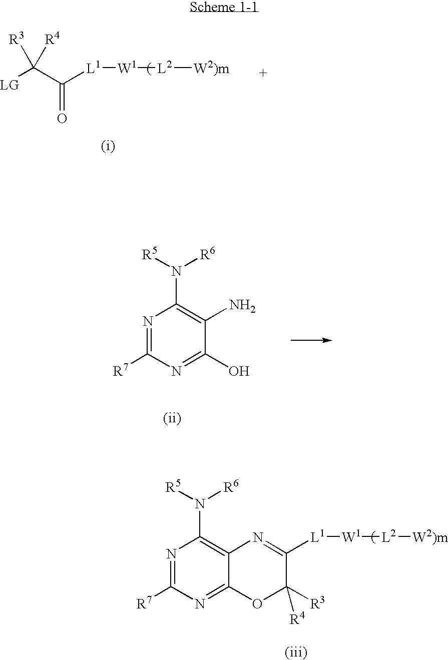
wherein LG is a leaving group (e.g., halogen atom, toluenesulfonate, methanesulfonate, trifluoromethanesulfonate and the like) and other symbols are as defined above.

As shown in Scheme 1-1, a compound of the formula (iii) can be prepared from a compound of the formula (ii) and a compound of the formula (i). Condensation of a compound of the formula (i) and a compound of the formula (ii) in an organic solvent or a mixed solvent thereof (including aqueous mixtures) in the presence or absence of an acid (e.g., HCl) or a base (e.g., NaHCO₃) provides, after workup, a compound of the formula (iii).

Reduction of the compound of the formula (iii) with a reducing agent, such as sodium borohydride, lithium borohydride and sodium triacetoxyborohydride, provides a compound of the formula (Iv), as shown in Scheme 1-2. 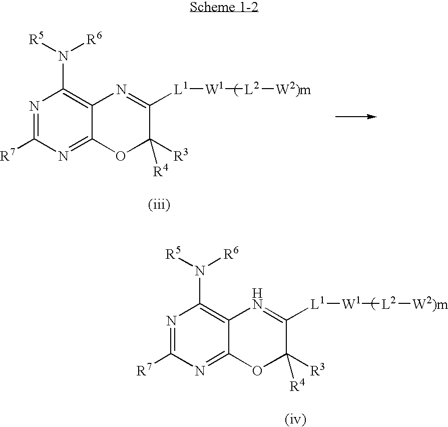
wherein each symbol is as defined above.

Schemes 1-3a-1-3i illustrate methods for the preparation of an intermediate compound of the formula (i). In Scheme 1-3a, a method for introducing a desired substituent onto the cyclohexane ring of a benzene derivative, which is a compound of the formula (v), is shown. A Horner-Emmons reaction or a similar Wittig reaction is used to introduce an α,β-unsaturated ester group, whereby a compound of the formula (vi) can be produced (e.g., reaction with a suitable phosphoric acid salt or phosphoric acid ester, in the presence of a base such as sodium hydride, in a solvent such as DMF or THF). Catalytic hydrogenation of a compound of the formula (vi) produces a compound of the formula (vii). For example, catalytic hydrogenation of a compound of the formula (vi) using a palladium or platinum catalyst in a relatively polar solvent such as THF, methanol, or an aqueous mixture containing an alcohol or THF as a co-solvent, is used to reduce the double bond, whereby a compound of the formula (vii) can be produced. A Friedel-Crafts acylation reaction of the compound of the formula (vii) is then used to attach a haloacetyl group on the benzene ring of the compound of the formula (vii), whereby a compound of the formula (ix) can be produced. Preferably, the leaving group in this series of reactions is Cl or Br. Suitable Lewis acids for the acylation include, for example, AlCl₃, AlBr₃, BCl₃, TiCl₄ and the like; suitable solvents are known in the art and include, for example, CS₂, nitrobenzene, dichloromethane, and similar solvents that are unreactive with the reagents and Lewis acids are employed. The production methods of intermediates are not limited to those mentioned above and synthesis methods known in the art are also employed. For example, acylation of a metalated aromatic compound, such as aryl lithium or aryl Grignard reagent, with an acylating agent such as N-methyl-N-methoxyamide of a chloroacetic acid derivative (commonly referred to as a Weinreb amide, see, Nahm and Weinreb (1981) Tetrahedron Lett. 22:3815-3818) or a suitable acyl ester, can be mentioned. Such methods afford production of other isomers of functionalized acetophenone derivatives. 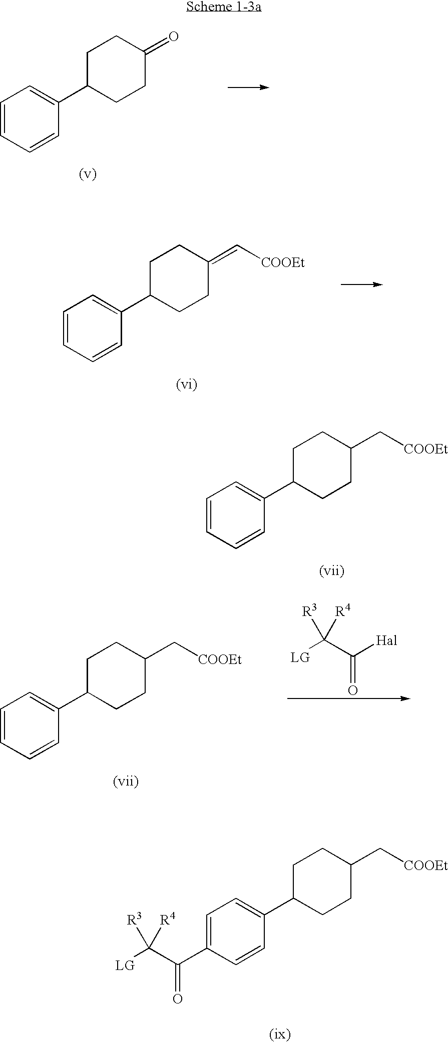
wherein Hal is halogen atom and other symbols are as defined above.

Alternatively, the compound of the formula (vii) may be alkylated by a treatment with a base such as lithium diisopropylamide or lithium hexamethyldisilazide in a suitable solvent such as THF, followed by a reaction with an alkylating agent such as alkyl halide, alkyl methanesulfonate, alkyl trifluoromethanesulfonate or alkyl toluenesulfonate, to give a compound of the formula (x) (Scheme 1-3b). If desired, the series of reactions may be repeated to give a compound of the formula (xi). Acylation of the compound of the formula (xi) can be accomplished as described above to give a compound of the formula (xii). 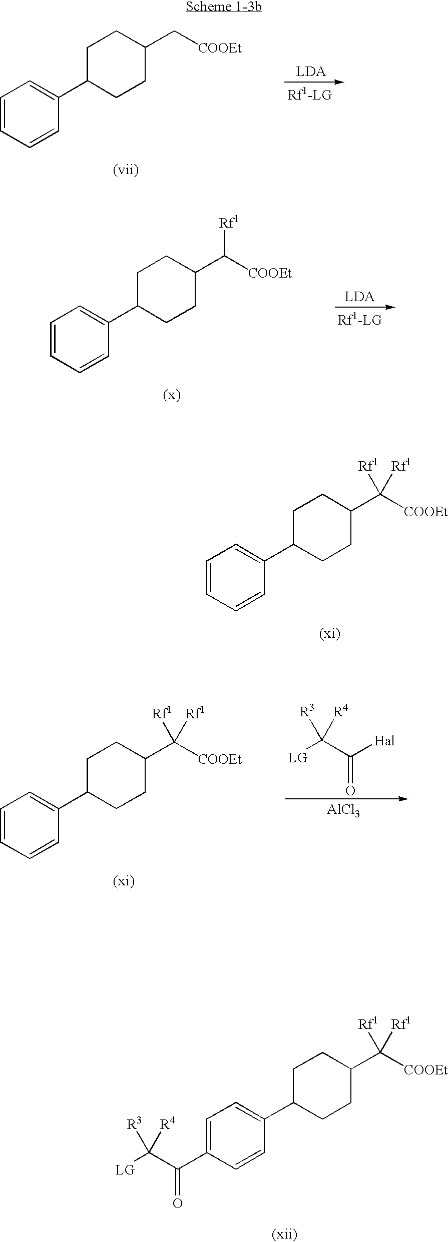
wherein each symbol is as defined above.

Similar approaches can be also used to produce other functionalized acetophenone derivatives, as shown in Scheme 1-3c. For example, the compound of the formula (v) can be converted into an aldehyde in two steps, for example, using a Wittig reaction with methoxymethyltriphenylphosphorane in a suitable solvent such as THF, DME or dioxane to produce a compound of the formula (xiv), followed by mildly acidic hydrolysis. This aldehyde can be converted to an α,β-unsaturated ester by a Wittig reaction with (carbomethoxy)methylenetriphenylphosphorane in a suitable solvent. If desired, the double bond can be reduced via catalytic hydrogenation using palladium on carbon to produce a compound of the formula (xv). Suitable solvents for hydrogenation reactions include ethanol, ethyl acetate and the like. Acylation of the compound of the formula (xv) to produce a compound of the formula (xvi) can be accomplished as described above for acylation of the compound of the formula (vii). 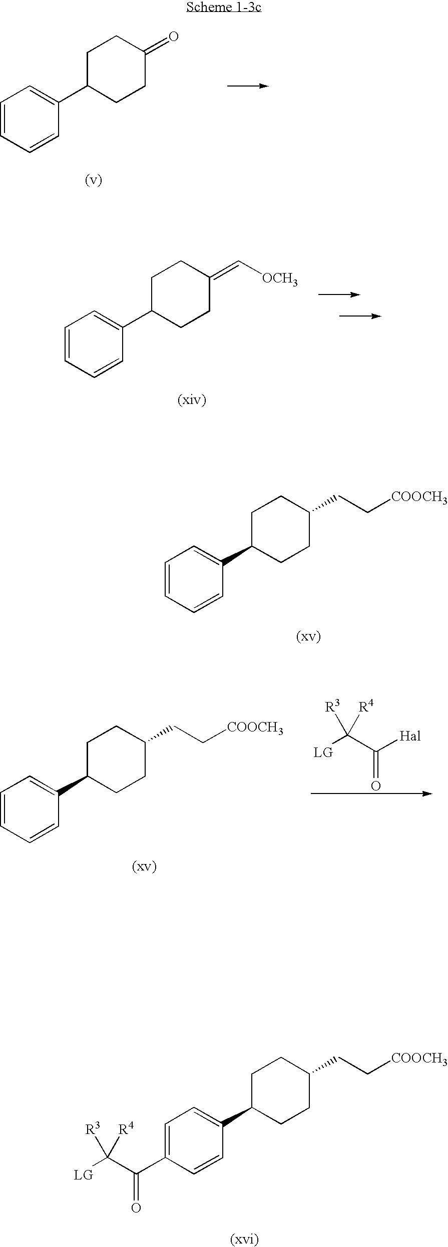
wherein each symbol is as defined above.

Scheme 1-3d illustrates production of acetophenone compound of the formula (i) other than the above, which is suitable for producing the compound of the formula (1). 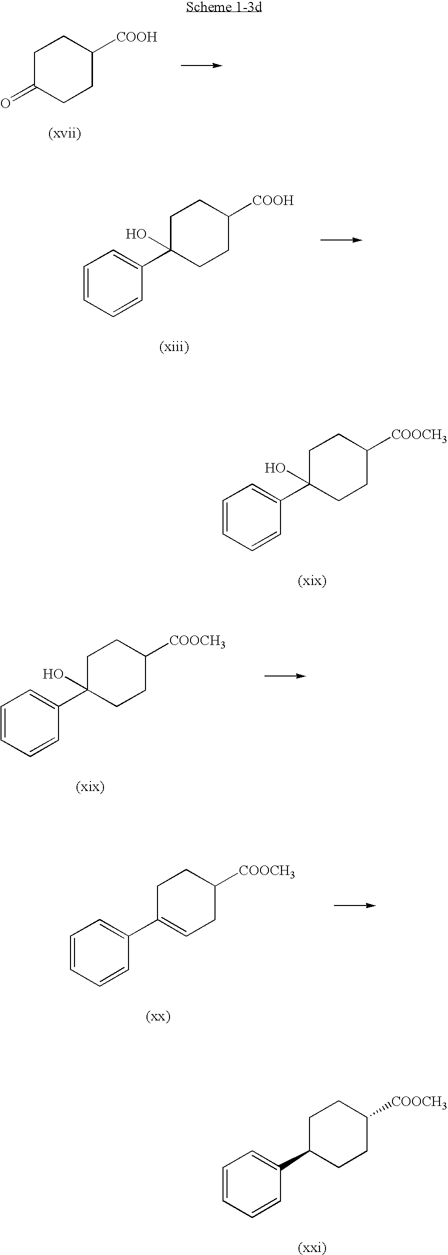 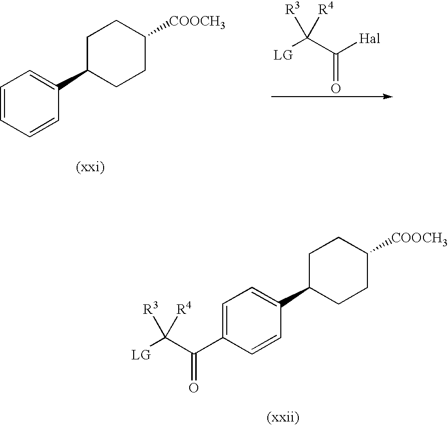
wherein each symbol is as defined above.

In Scheme 1-3d, a phenyl group is introduced onto compound (xvii) using, for example, a phenyl Grignard reagent or phenyl lithium to provide a compound of the formula (xiii). The carboxylic acid can be esterified under conventional conditions to produce a compound of the formula (xix), and dehydration can be accomplished using an acid catalyst such as acetic acid, hydrochloric acid or trifluoroacetic acid in a suitable solvent such as chloroform or toluene to produce a compound of the formula (xx). Reduction of the cyclohexene double bond can be performed under catalytic hydrogenation conditions using palladium as a conventional catalyst, thereby to provide a compound of the formula (xxi). This reduction produces a mixture of isomers (both cis and trans-disubstituted cyclohexanes are produced). Where desired, these can be isomerized using a base such as alkoxide or DBU in methanol or toluene to primarily produce a more thermodynamically stable trans-disubstituted isomer. Acylation of the compound of the formula (xxi) to produce a compound of the formula (xxii) is performed as described above.

The compounds of the formula (1) that contain a heterocyclic ring for W² can be synthesized by a method similar to the above except that the heterocyclic ring is stable under the acylation reaction conditions. For example, a compound where W² is an acylated piperidine can be produced by a series of reactions shown in Scheme 1-3e. In this series of reactions, the compound of the formula (xxiii) is alkylated, sulfonylated or acylated on the nitrogen atom using reagents and conditions known in the art (exemplified below), e.g., acylation with diethyl oxalate or ethyl oxalyl chloride in the presence of a mild base such as triethylamine or pyridine) to produce a compound of the formula (xxiv). The compound of the formula (xxiv) is then dehydrated and catalytically reduced as described above in Scheme 1-3d to give a compound of the formula (xxv). This compound is then acylated as described above to give a compound of the formula (xxvi). 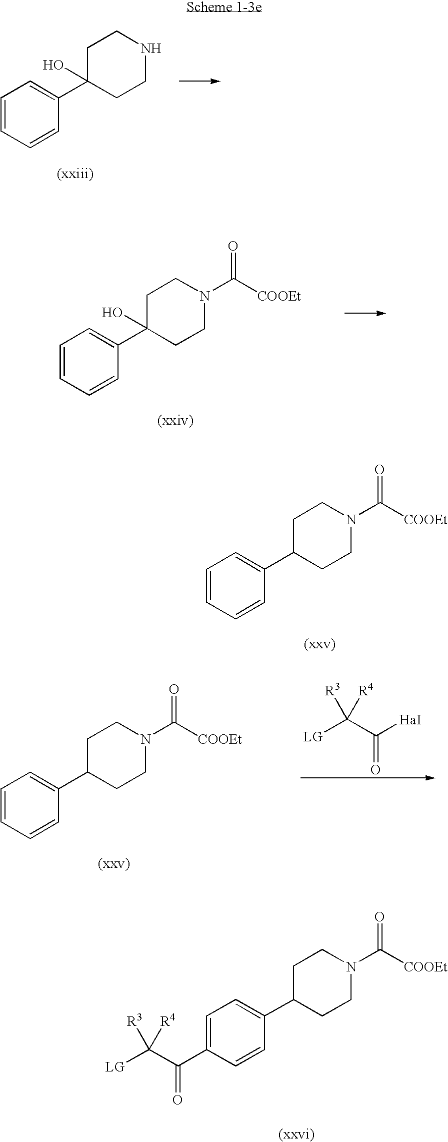
wherein each symbol is as defined above.

Similarly, the compound of the formula (xxvii) can be alkylated, sulfonylated or acylated on the nitrogen atom to give a compound of the formula (xxviii), which is then acylated as described above to produce a compound of the formula (xxix). 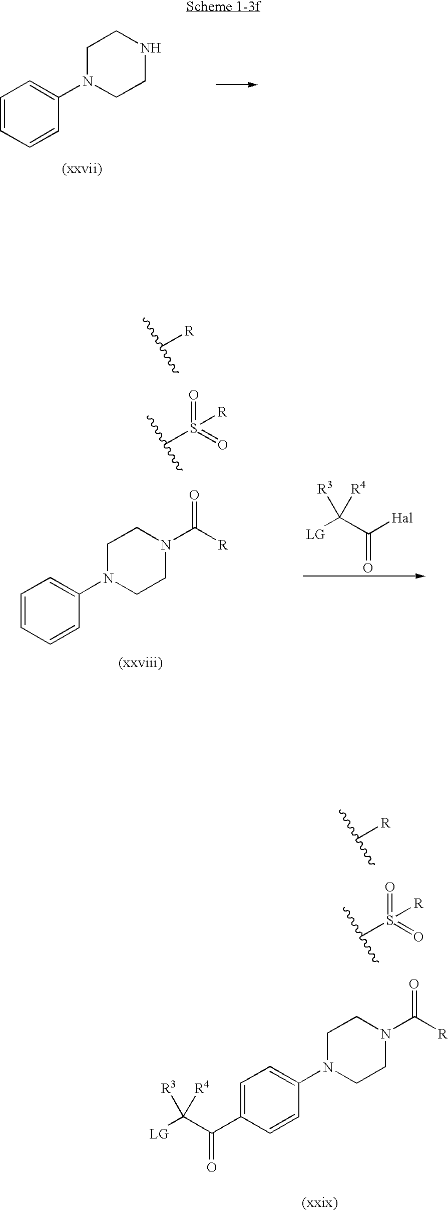
wherein R is as defined for R^{d1} or R^{e1}, and other symbols are as defined above.

Other compounds of the formula (1) having a heterocycle as W² can be produced as shown in Scheme 1-3g. For example, a compound of formula (i) can be produced by attaching a heterocyclic group onto acetophenone and then halogenating the acetophenone at the α carbon. In this series of reactions, a compound of the formula (xxx) is synthesized by acylation of fluorobenzene under typical Friedel-Crafts conditions as described above. The 4-fluoro group is then subjected to aromatic nucleophilic displacement reactions. For example, as shown in the scheme, it is displaced by a substituted piperidine group by reaction with nucleophilic piperidine in a polar aprotic solvent such as DMSO or DMF. Then, compounds of the formula (xxxii) can be producted by, for example, using bromine (Br₂) or chlorine (Cl₂) in a polar solvent such as DME or ethyl acetate, in the presence of an acid catalyst such as acetic acid or hydrobromic acid. 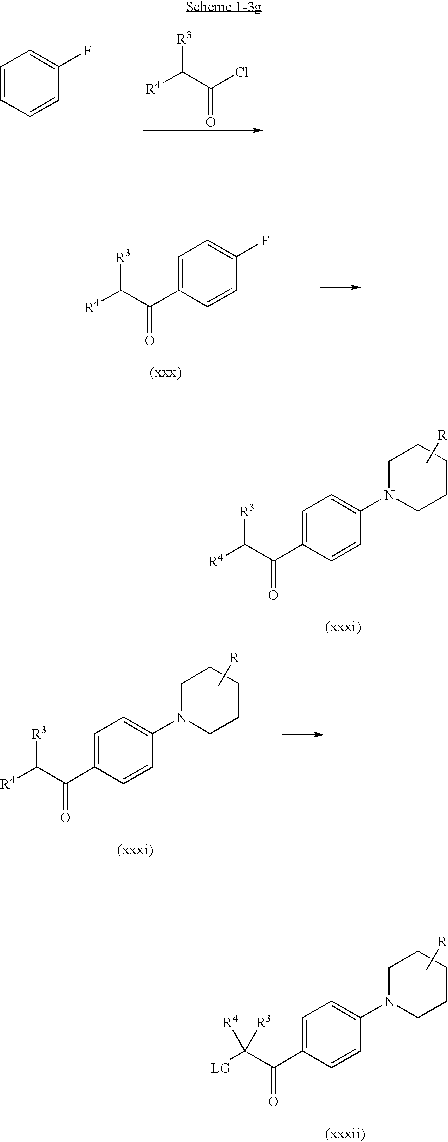
wherein R is oxo, halogen atom, R^{h1}, —OR^{h1}, —N(R^{h1})₂, —(CH₂)t—S(O)uR^{e1}, cyano group, nitro group, C₁₋₈ haloalkyl group, C₁₋₈ haloalkoxy group, aryl C₁₋₄ alkyl group, heteroaryl C₁₋₄ alkyl group, —CH(R^{fl})—CO₂R^{e1}, —C(R^{f1})₂—CO₂R^{e1}, —C(O)CO₂R^{e1}, ═CH—CONR^{e1}R^{f1}, ═CH—CO₂R^{e1}, —(CH₂t—CO₂R^{e1}, —(CH₂) t-C(O)R^{e1}, —(CH₂)t—C(O)NR^{e1}R^{f1}, —(CH₂)t—NHSO₂R^{e1}, —(CH₂)t-SO₂NR^{e1}R^{f1}, (CH₂)t-NR^{e1}R^{f1}, —(CH₂)t—OR^{e1}, (CH₂)t-NHSO₂NHCO₂R^{e1}, —(CH₂)t—NHSO₂NR^{e1}R^{f1}, —(CH₂)t—CONHSO₂R^{e1}, —(CH₂)t-W³, —(CH₂) t-NHCO₂R^{e1}, —(CH₂)t-NR^{f1}COR^{e1}, —(CH₂)t-NHCONR^{e1}R^{f1}, —(CH₂)t—NHCO—(CH₂)t—OCOR^{e1}, wherein R^{h1} is as defined for R^{c1}, and other symbols are as defined above.

Acetophenone derivatives having a functional group suitable for the preparation of the compounds of formula (1) where L¹ is a single bond can be prepared from substituted acetophenone, especially when R³ and R⁴ are identical groups, as shown in Scheme 1-3h. For example, a compound of the formula (xxxiii) can be alkylated with an alkylating agent such as methyl iodide, ethyl bromide or other similar alkylating agent in the presence of a base such as lithium diisopropylamide, lithium hexamethyldisilazide or sodium hydride, in a solvent such as DMF, DME, THF or toluene. This produces a compound of the formula (xxxiv) where R³ and R⁴ are the same. This compound can then be halogenated as described above in Scheme 1-3g to produce a compound of the formula (i) (compound of the formula (xxxv)), which is then condensed with substituted pyrimidine as shown in Scheme 1-1 to prepare the compounds of the formula (1). 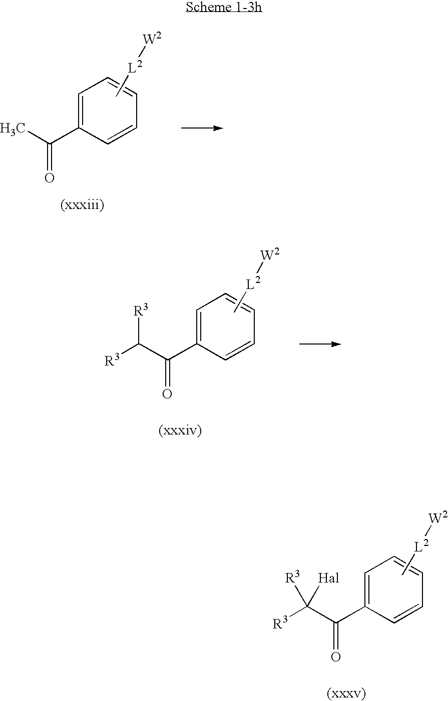
wherein each symbol is as defined above.

The compounds of the formula (1) wherein W¹ contains a heterocyclic ring can be synthesized using similar procedures, as outlined in Scheme 1-3g. For example, a compound of the formula (xxxvi) such as furan, thiophene, pyrrole, oxazole, thiazole, imidazole or thiadiazole can be lithiated with butyl lithium or lithium diisopropylamide in a suitable solvent such as THF, DME or dioxane. The lithiated compound may be reacted with, for example, an amide, such as an dimethylamide or an N-methyl-N-methoxyamide to produce a compound of the formula (xxxvii), which in turn halogenated as described above to produce a compound of the formula (xxxviii). In a similar sequence, a compound of the formula (xxxix) can be lithiated and acylated to give a compound of the formula (xl), which in turn can be halogenated as described above. Other heterocycles may be employed for these conversion. 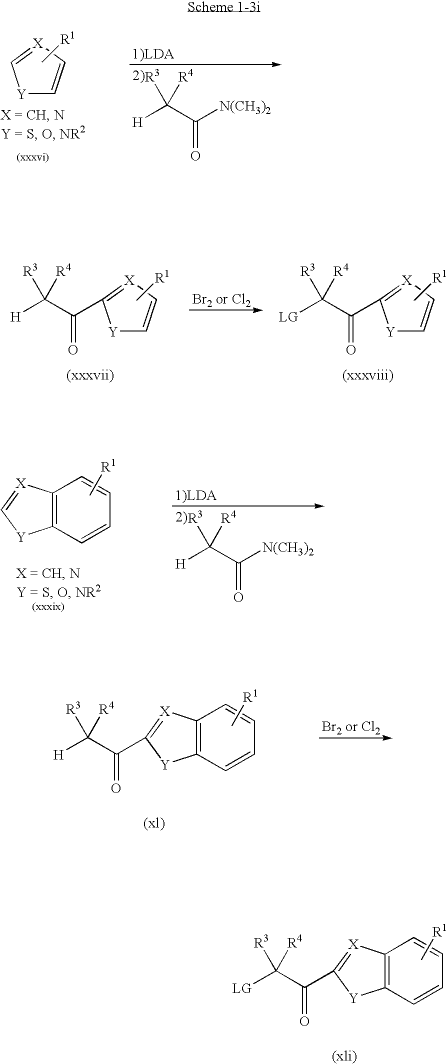
wherein each symbol is as defined above.

As shown in Scheme 1-4, compounds of the formula (Iv) having a substituted phenyl group for W¹ and a substituted cyclohexane ring for L²—W² (e.g., a compound of the formula (xlii)) can be prepared from a compound of the formula (ii) and a compound of the formula (ix). 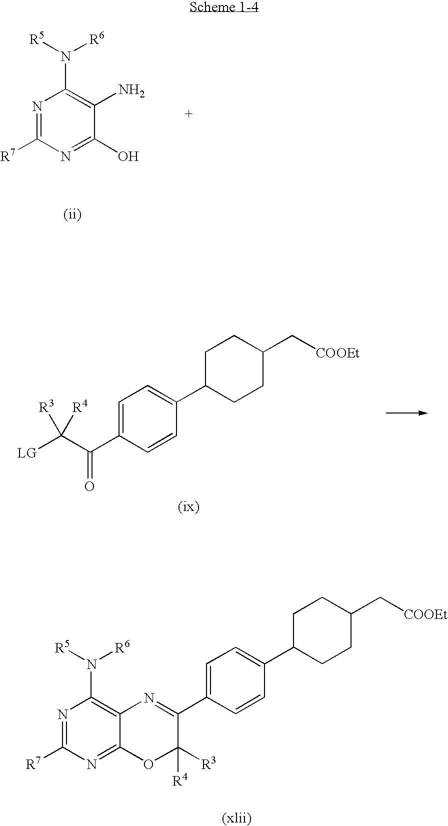
wherein each symbol is as defined above.

The compound of the formula (xlii) can be used to make other compounds of the formula (1). For example, a compound of the formula (xliii) can be produced by hydrolysis of the compound of the formula (xlii) (Scheme 1-5). Ester hydrolysis can be accomplished in any solvent that can dissolve the compound of the formula (xlii) and is at least partially miscible with water, by treating a solution of the compound of the formula (xlii) with an aqueous base such as sodium hydroxide or potassium hydroxide. The carboxyl group can be converted to other groups such as amide group by the methods known in the art. For example, carboxylic acid can be activated by condensation with a variety of coupling reagents such as hydroxybenzotriazole (HOBt) and N-hydroxysuccinimide (HOSu), using dicyclohexylcarbodiimide (DCC) or a similar carbodiimide reagent or a wide variety of reagents used for formation of peptide bonds. Conditions for these reactions are those known in the art. The activated intermediate such as an ester of HOBt or HOSu can then be condensed with a wide variety of nucleophiles such as amines, alcohols and thiols. Scheme 1-5 shows conversion of a compound of formula (xlii) to a compound of the formula (xliv) by this sequence using ammonia as the nucleophile. 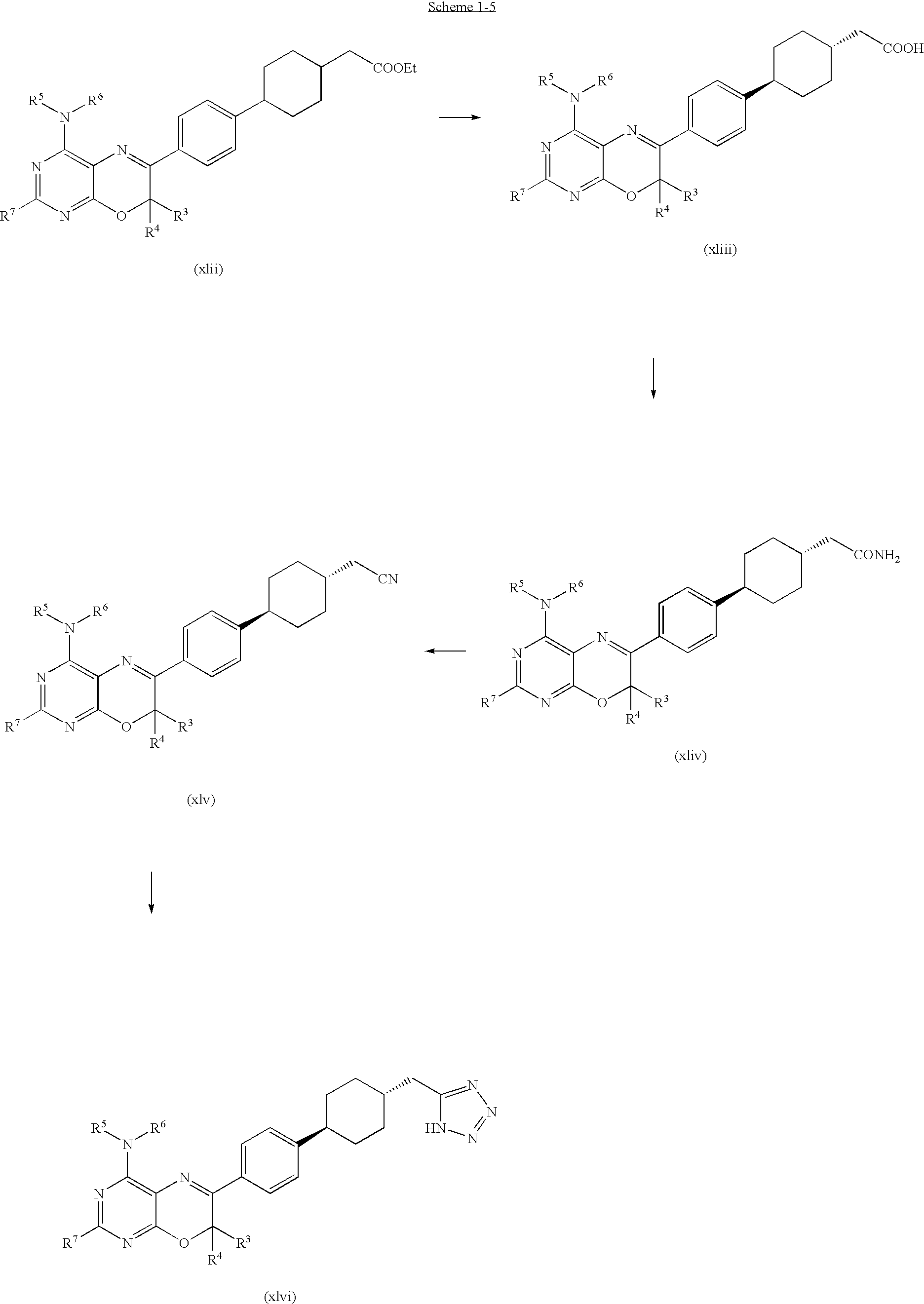
wherein each symbol is as defined above.

Dehydration of the compound of the formula (xliv) to give a compound of the formula (xlv) can be accomplished by a variety of methods. See Scheme 1-5 above. Phosphorous pentoxide is the most common dehydrating reagent for this reaction, but many other reagents known in the art can be used. The cyano group of the compound of the formula (xlv) can be converted to other groups such as a tetrazolyl group by the methods known in the art to produce a compound of the formula (xlvi). For example, this conversion can be carried out by reacting the nitrile with azide such as sodium azide or lithium azide, or hydrazoic acid in a solvent such as DMF or water.

Schemes 1-6a and 1-6b illustrate one approach to the preparation of a compound of the formula (1) wherein W¹ is phenylene having an additional substituent other than L²—W². As shown in Scheme 1-6a, a compound of the formula (xlvii) can be nitrated under usual conditions (using a dehydrating agent such as nitric acid, in the presence of sulfuric acid in a solvent such as chloroform, methylene chloride, acetic acid, or neat) to provide a compound of the formula (xlviii). Reduction of the nitro group is associated with debromination using catalytic hydrogentation or SnCl₂ (generally in alcoholic solvents) to provide a compound of the formula (xlix). Chloride replacement of the amino group is accomplished using copper chloride in the presence of a suitable nitrite (e.g., tert-butyl nitrite, sodium nitrite) and a solvent, whereby a compound of the formula (1) is provided. Bromine can be re-introduced under standard brominating conditions (e.g., Br₂, N-bromosuccinimide or CuBr₂), providing a compound of the formula (li). Alternatively, the compound of the formula (xlvii) can be directly chlorinated using a conventional reagent (e.g., sulfuryl chloride, Cl₂ or N-chlorosuccinimide) under conditions known in the art to provide a compound of the formula (li). 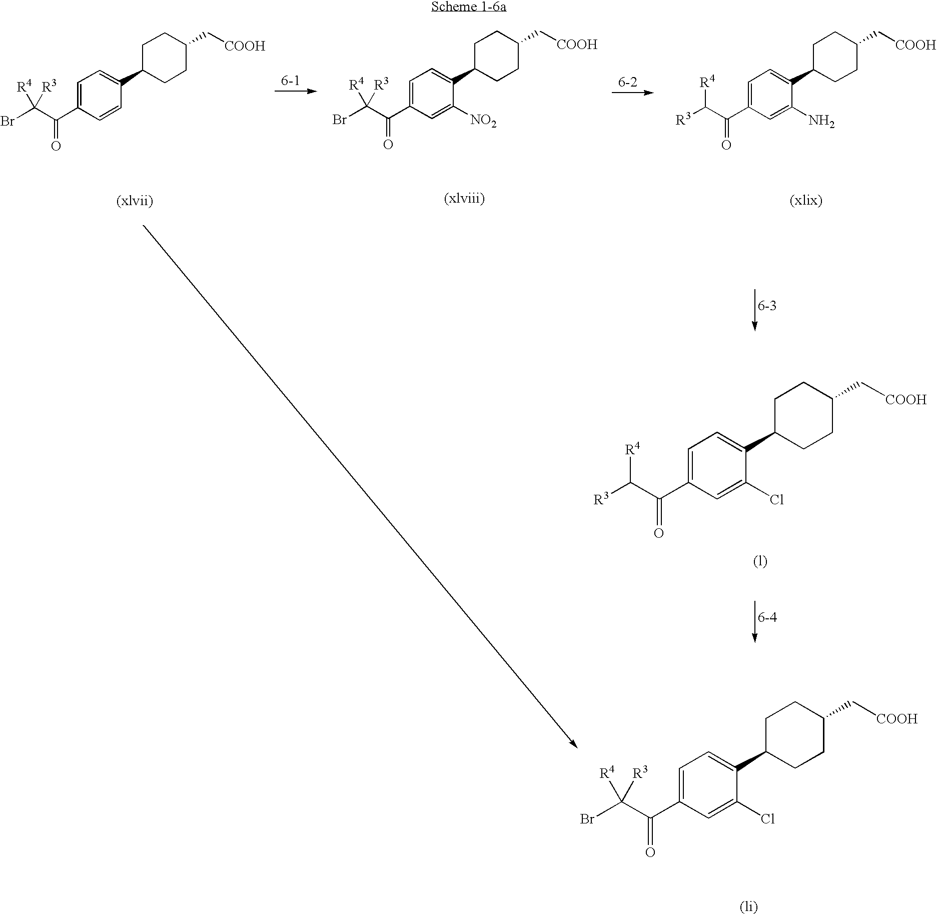
wherein each symbol is as defined above.

Scheme 1-6b illustrates production of other compounds from the compound of the formula (xlix). For example, a compound of the formula (lii) (wherein X¹⁰ is F) can be produced from the compound of the formula (xlix) using a fluorinating reagent such as nitrosonium tetrafluoroborate, DAST, HF or CsF (generally in a solvent such as toluene, benzene, methylene chloride or dichloroethane). Subsequent bromination of the compound of the formula (lii) to produce a compound of the formula (liii) can be accomplished according to known methods. Conversion of the compound of the formula (li) or (liii) to a compound of the formula (liv) is accomplished via condensation with suitably substituted pyrimidine. 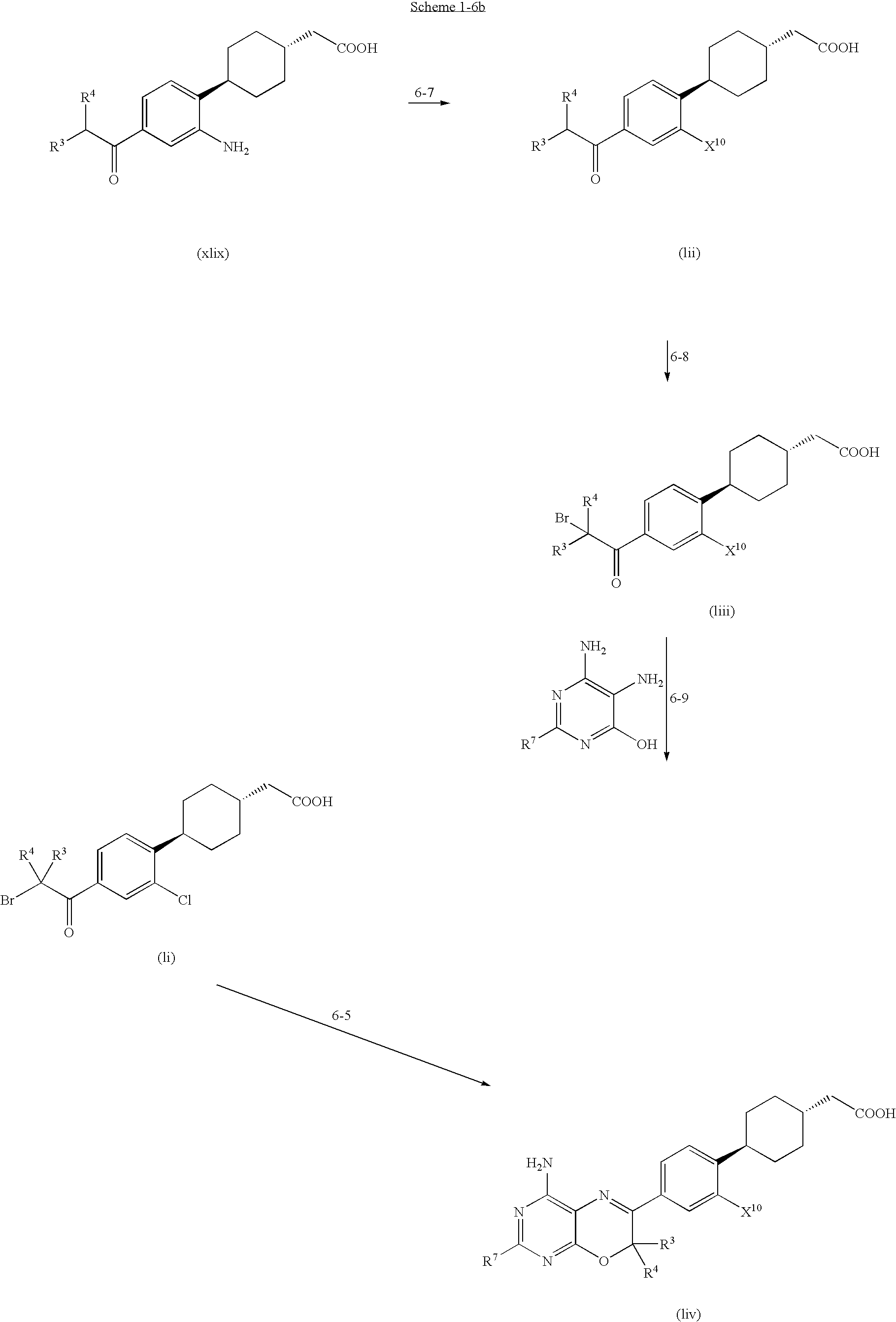
wherein each symbol is as defined above.

As shown in Scheme 1-7, a compound of the formula (1) wherein X is N, Y is CH, Z is O, L¹ is a single bond and W¹ is an optionally substituted arylene or heteroarylene can be prepared by, for example, a palladium-catalyzed cross coupling reaction of the compound of the formula (Iv) and a compound of the formula (lvi). 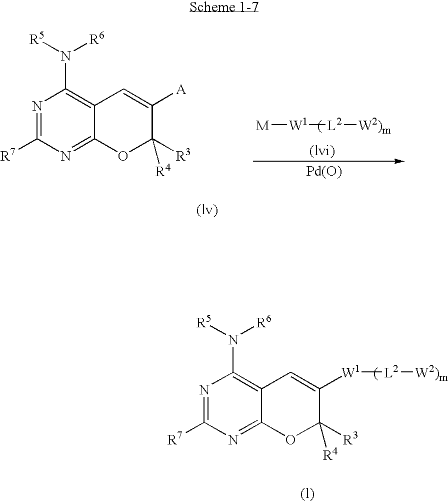
wherein A is a halogen, e.g., Br, I or triflate or other suitable substituent known in the art and M is B(OR^{X}), Sn(R^{Y}) or other suitable metal known in the art. A and M are also interchangeable.

Scheme 1-8 illustrates a method for the preparation of a compound of the formula (lv). Condensation of the compound of the formula (lviii) with a compound of the formula (lvii) in a suitable solvent such as acetic acid affords a compound of the formula (lix). Conversion of the hydroxy moiety to a leaving group, such as a chloride atom or bromide atom with phosphorus oxychloride or phosphorus oxybromide, respectively, is followed by displacement of the leaving group with —NR⁵R⁶ to afford a compound of the formula (Iv). 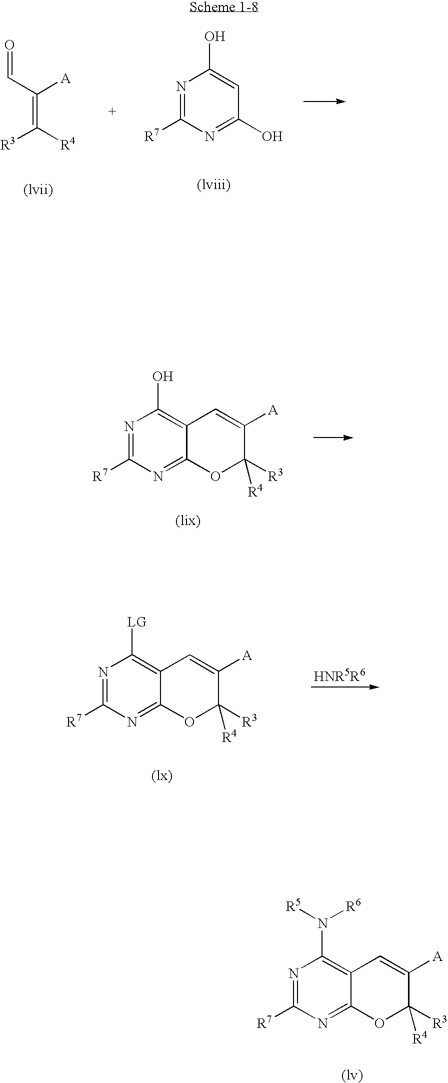
wherein each symbol is as defined above.

A compound of the formula (2) can be prepared from commercially available starting materials using synthetic techniques known in the art. The production method of a compound of the formula (2), for example, wherein R⁵′ is a hydrogen atom, is shown in Scheme 2-1. 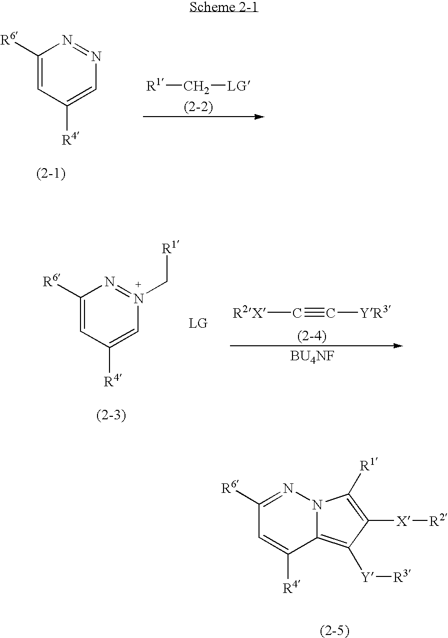
wherein LG′ is a leaving group (e.g., halogen atom, toluenesulfonate, methanesulfonate, trifluoromethanesulfonate and the like) and other symbols are as defined above.

As shown in Scheme 2-1, a compound of the formula (2-5) can be prepared by reacting a compound of the formula (2-3) with a compound of the formula (2-4). Condensation of the compound of the formula (2-3) and the compound of the formula (2-4) in an organic solvent or a mixture thereof (including aqueous mixtures) in the presence of a base (e.g., tetrabutyl ammonium fluoride) provides, after workup, a compound of the formula (2-5). The compound of the formula (2-3) can be obtained by a treatment of the compound of the formula (2-1) with the compound of the formula (2-2), which is an alkylating agent, in an organic solvent or a mixture thereof. The compound of the formula (2-1) can be obtained by the methods of Schemes 2-2 to 2-4.

In Scheme 2-2, the compound of the formula (2-6) is condensed with glyoxylic acid under basic conditions to provide a compound of the formula (2-7). Subjecting the compound of the formula (2-7) to a treatment with an acid such as HCl or H₂SO₄/HCO₂H results in the production of a compound of the formula (2-8) (see, e.g., Dean et al. (1993) J. Org. Chem. 58:7916-7917). Treatment of the compound of the formula (2-8) with hydrazine results in the production of a compound of the formula (2-9), which can be converted to a compound of the formula (2-10) by a treatment with a chlorinating agent such as POCl₃, PCl₃, PCl₅ or SOCl₂. The compound of the formula (2-10) can be treated with nucleophile, e.g., amine, to provide a compound of the formula (2-11). Catalytic hydrogenation of the compound of the formula (2-11) using a palladium or platinum catalyst in a relatively polar solvent such as THF, methanol or an aqueous mixture containing an alcohol or THF as a co-solvent can be used to remove the halogen atom, producing a compound of the formula (2-12). 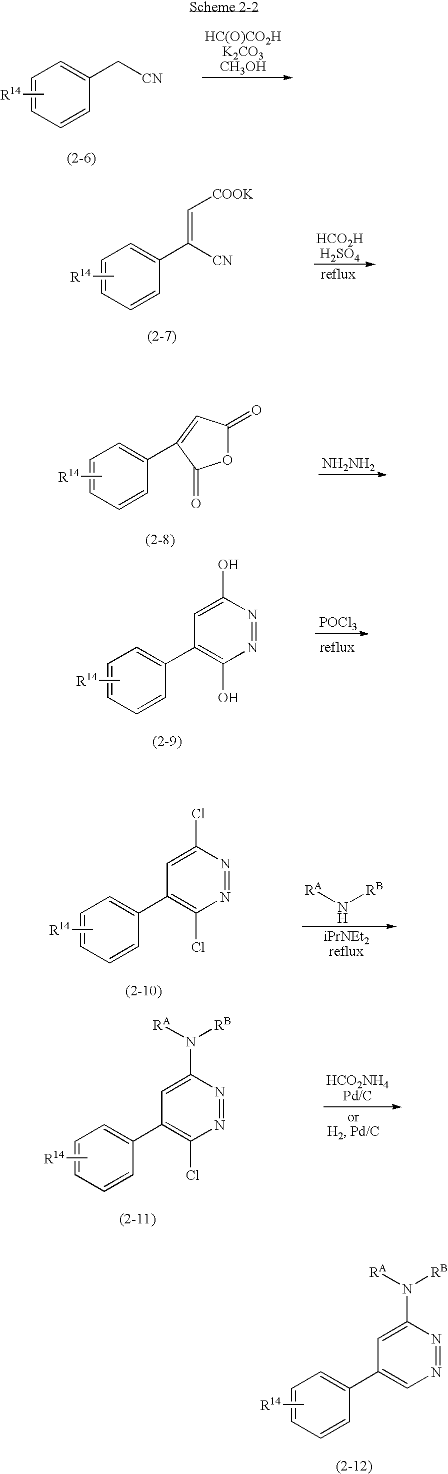
wherein R¹⁴ is C₁₋₄ alkyl group, C₁₋₈ fluoroalkyl group, halogen atom or aryl group, R^{A} and R^{B} are the same or different and each is hydrogen atom, C₁₋₈ alkyl group, C₂₋₈ alkenyl group, C₂₋₈ alkynyl group, C₁₋₈ fluoroalkyl group, aryl group, aralkyl group or C(O)Rt wherein Rt is hydrogen atom, C₁₋₈ alkyl group, amino group, C₁₋₄ alkylamino group, di(C₁₋₄ alkyl)amino group, aralkyl group or C₁₋₈ alkoxy group.

Scheme 2-3 illustrates production of the compounds of the formulas (2-13) and (2-14) from the compound of the formula (2-10) in the same manner as in the production method of the compounds of the formulas (2-11) and (2-12) except the use of R^{A}OH instead of NH(R^{A}) (R^{B}). 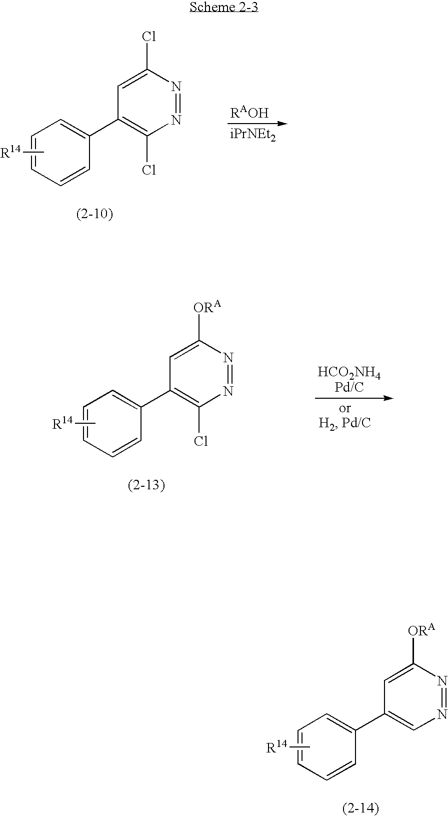
wherein each symbol is as defined above.

A different approach as shown in Scheme 2-4 can be used For example, the compound of the formula (2-15) can be alkylated in aqueous sulfuric acid with silver (I) peroxydisulfate in the presence of carboxylic acid to afford a compound of the formula (2-16) (see, e.g., Samaritoni (1998) Org. Prep. Proced. Int. 20:117). Conversion of the compound of the formula (2-16) to a compound of the formula (2-18) can be accomplished as described above for the conversion of the compound of the formula (2-10) to the compound of the formula (2-12). Similarly, the compound of the formula (2-16) can be converted to a compound of the formula (2-20) as described above for the conversion of the compound of the formula (2-10) to the compound of the formula (2-14). 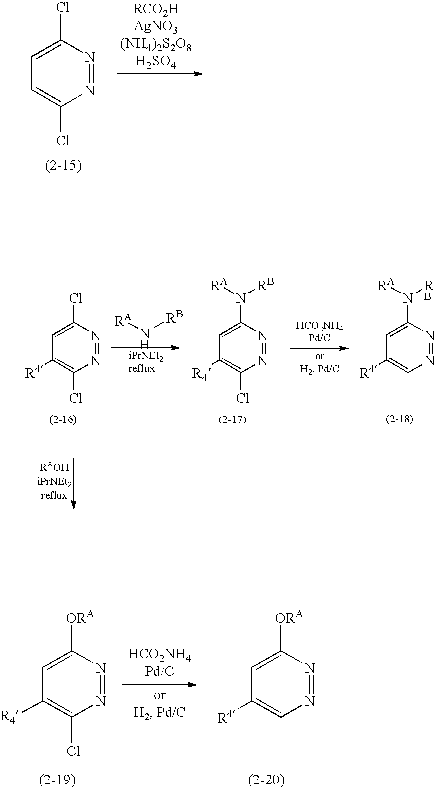
wherein each symbol is as defined above.

The compound of the formula (2) can be also produced by the method shown in Schemes 2-5 to 2-8. Scheme 2-5 illustrates that the compound of the formula (2-22) or (2-23) can be produced by hydrolysis of one of or both ester groups of the compound of the formula (2-21). Ester hydrolysis can be accomplished in any solvent that dissolves the compound of the formula (2-21) or is at least partially miscible with water, by treating a solution of the compound of the formula (2-21) with an aqueous base such as lithium hydroxide, sodium hydroxide or potassium hydroxide. 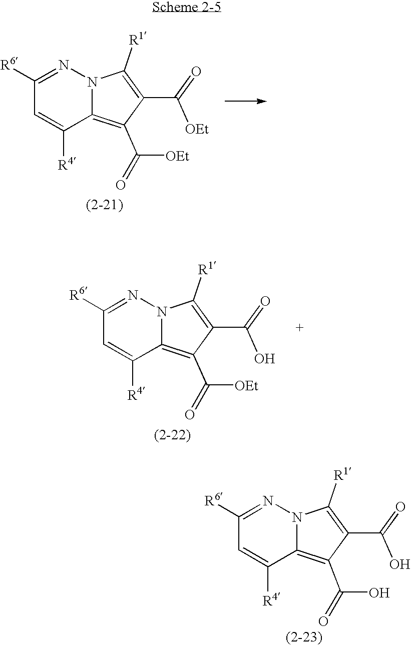
wherein each symbol is as defined above.

The carboxylic acid can be converted to other groups by the methods known in the art. Scheme 2-6 illustrates one method for the conversion of a compound of the formula (2-22) to a compound of the formula (2-25). The conversion method is not limited to this method and a different method known in the art can be also employed. For example, the compound of the formula (2-22) can be converted to a compound of the formula (2-24) via a Curtius rearrangement (see, e.g., March, J. Advanced Organic Chemistry, 4^{th} ed., John Wiley & Sons: New York, 1992; pp 1091-1092). Treatment of the compound of the formula (2-24) with chloroformate in the presence of a base (typically tertiary amine) produces a compound of the formula (2-25).

The compound of the formula (2-22) can be activated by condensation with a variety of coupling reagents such as hydroxybenzotriazole (HOBt) and N-hydroxysuccinimide (HOSu), using dicyclohexylcarbodiimide (DCC) or a similar carbodiimide reagent, or a wide variety of reagents used for the formation of peptide bonds. Conditions for such reactions are well known in the art. The activated intermediate such as an ester of HOBt or HOSu can be condensed with a wide variety of nucleophiles such as amines, alcohols and thiols, to produce other esters, thioesters or amides. Scheme 2-6 shows the conversion of the compound of the formula (2-22) to an amide compound of the formula (2-26) by this sequence using ammonia as nucleophile. Dehydration of the compound of the formula (2-26) can be accomplished by a variety of methods. Phosphorous pentoxide is the most common dehydrating reagent for this reaction, but many others known in the art can be used. The cyano group of the compound of the formula (2-27) can be converted to other groups such as a tetrazolyl group (the compound of the formula (2-28)) by the methods known in the art. For example, this conversion can be performed by reacting the nitrile with azide such as sodium azide or lithium azide, or hydrazoic acid in a solvent such as DMF or water. 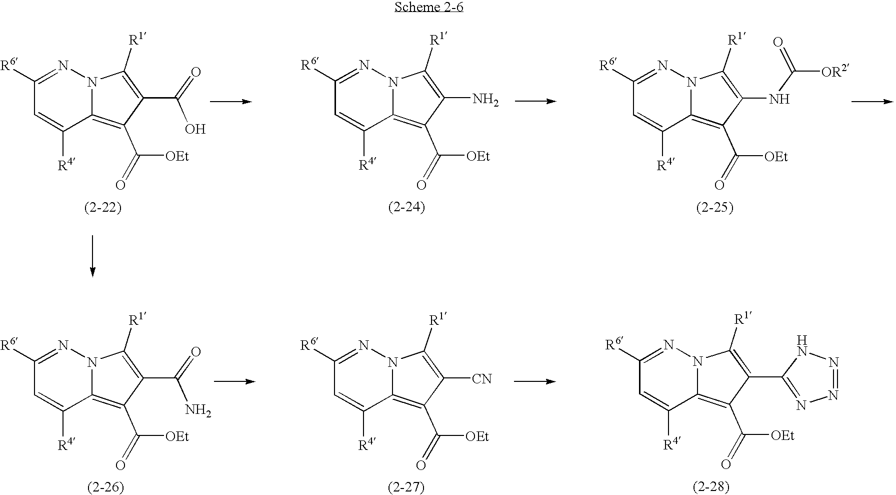
wherein each symbol is as defined above.

Additional examples of the conversion of the compound of the formula (2-22) to other compounds are shown in Scheme 2-7. 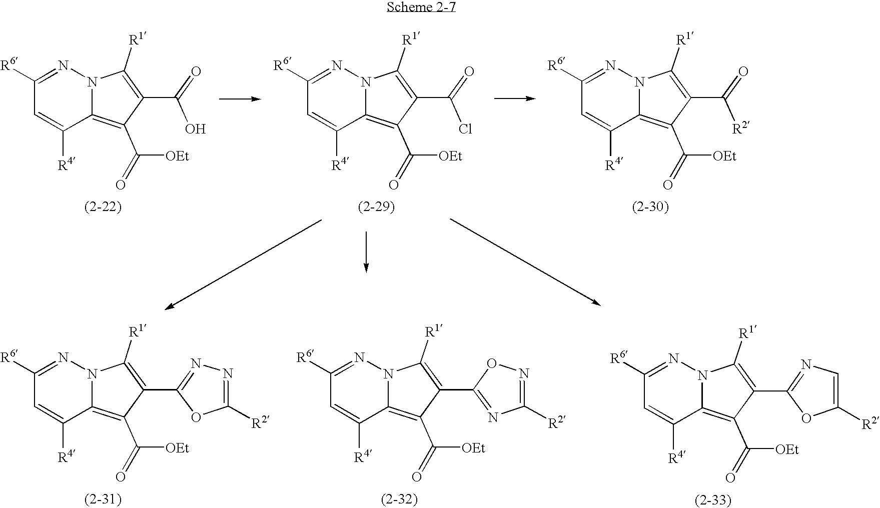
wherein each symbol is as defined above.

Conversion of the compound of the formula (2-22) to a compound of the formula (2-29) can be accomplished using a reagent such as oxalyl chloride, POCl₃, PCl₃, PCl₅ or SOCl₂. The compound of the formula (2-29) can be treated with, for example, a lithium dialkylcopper reagent to give a compound of the formula (2-30). The compound of the formula (2-29) can also be used to produce a heterocyclic derivative such as [1,3,4]-oxadiazole compounds (compound of the formula (2-31)), [1,2,4]-oxadiazole compounds (compound of the formula (2-32)) and oxazole compounds (compound of the formula (2-33)), using the methods known in the art. For example, reacting the compound of the formula (2-29) with acyl hydrazide in the presence of a base such as triethylamine, followed by a treatment with P₄O₁₀ at an elevated temperature accomplishes the conversion to a compound of the formula (2-31). In another example, the compound of the formula (2-29) can be reacted with N-hydroxyamidine in the presence of a base, and the product treated with tetrabutylammonium fluoride to give a compound of the formula (2-32). In yet another example, the compound of the formula (2-29) can be treated with an α-aminoketone in the presence of a base such as triethylamine or pyridine, and subsequently applied to dehydrating conditions with, for example, sulfuric acid, P₄O₁₀ or PPh₃-diethyl azodicarboxylate to produce a compound of the formula (2-33).

Compounds of the formula (2) other than the above can be prepared from the compound of the formula (2-21), as illustrated in Scheme 2-8. For example, reduction of the compound of the formula (2-21) with a reagent such as LiAlH₄ or LiBEt₃H in a suitable solvent (e.g., THF, diethyl ether or dimethoxyethane) produces a compound of the formula (2-34). The compound of the formula (2-34) can be alkylated or acylated by known methods to give a compound of the formula (2-35) or a compound of the formula (2-36), respectively. 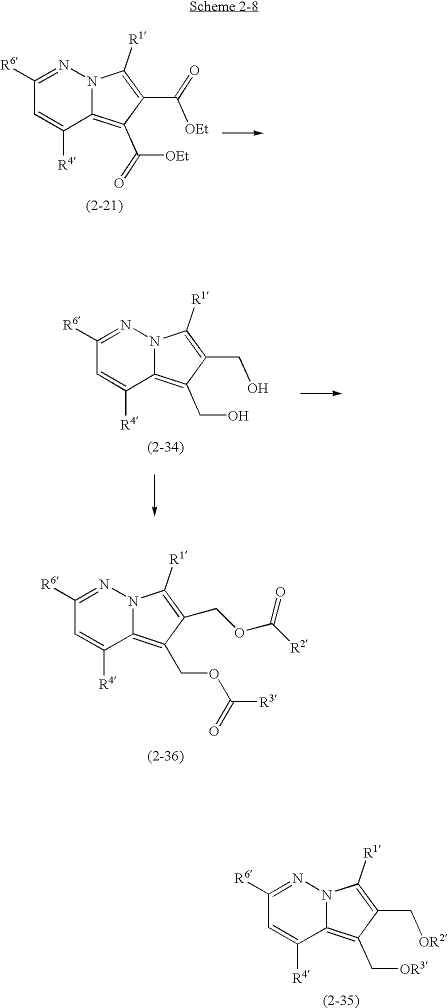
wherein each symbol is as defined above.

The compounds represented by the above-mentioned formulas (3)-(6) can be produced according to the methods disclosed in JP-A-H5-213985, JP-A-H8-182496, WO00/58491 and JP-A-2004-67635, respectively.

When the present invention is used as an anorectic or a therapeutic agent for obesity, hyperlipidemia, diabetes, arteriosclerosis, coronary disease and hypertension, it is systemically or topically administered orally or parenterally. While the dose varies depending on age, symptoms, treatment effect and the like, it is generally administered at a dose of 1 mg-1 g once or several times a day for an adult.

A compound having a DGAT inhibitory activity (e.g., DGAT1 inhibitory activity) can be admixed with a suitable diluent, powder, adsorbent, solubilizer and the like to process into a solid composition or a liquid composition for oral administration, or a preparation for parenteral administration such as injection and the like.

In addition, a compound having a DGAT inhibitory activity (e.g., DGAT1 inhibitory activity) can be used for the treatment or prophylaxis of obesity, hyperlipidemia, diabetes, arteriosclerosis, coronary disease and hypertension in human as well as animals (for example, mammal) other than human, or as an anorectic.

A compound having a DGAT inhibitory activity (e.g., DGAT1 inhibitory activity) can be used concurrently with one or more other pharmaceutical agents according to conventional methods employed for pharmaceutical agents. There are various pharmaceutical agents that can be used concurrently with a compound having a DGAT inhibitory activity (e.g., DGAT1 inhibitory activity). When, for example, a compound having a DGAT inhibitory activity (e.g., DGAT1 inhibitory activity) is used as a therapeutic agent for obesity, it can be used in combination with other therapeutic agents for obesity. By the other therapeutic agents for obesity is meant compounds other than a compound having a DGAT inhibitory activity (e.g., DGAT1 inhibitory activity), which are generally used as therapeutic agents for obesity. Examples thereof include mazindol, orlistat, sibutramine and the like.

When a compound having a DGAT inhibitory activity (e.g., DGAT1 inhibitory activity) is used as a therapeutic agent for hyperlipidemia, it can be used in combination with other therapeutic agents for hyperlipidemia. By the other therapeutic agents for hyperlipidemia is meant compounds other than a compound having a DGAT inhibitory activity (e.g., DGAT1 inhibitory activity), which are generally used as therapeutic agents for hyperlipidemia. Examples thereof include statin drugs, fibrate drugs, probucol, nicotinic acid, cholesterol absorption suppressants, MTP inhibitors, ACAT inhibitors and CETP inhibitors. As the statin drugs, for example, lovastatin, simvastatin, pravastatin, fluvastatin, atorvastatin, cerivastatin, pitavastatin, nisvastatin, rosuvastatin and the like can be mentioned, and one or more thereof can be combined. As the fibrate drugs, for example, clofibrate, clinofibrate, sinfibrate, fenofibrate, bezafibrate, gemfibrozil and the like can be mentioned, and one or more thereof can be combined. As the cholesterol absorption suppressants, for example, ezetimibe, colestimide, colestyramine, colestipol and the like can be mentioned, and one or more thereof can be combined.

When a compound having a DGAT inhibitory activity (e.g., DGAT1 inhibitory activity) is used as a therapeutic agent for diabetes, it can be used in combination with other therapeutic agents for diabetes. By the other therapeutic agents for diabetes is meant compounds other than a compound having a DGAT inhibitory activity (e.g., DGAT1 inhibitory activity), which are generally used as therapeutic agents for diabetes. Examples thereof include insulin preparations, sulfonylureas, insulin secretagogues, sulfonamides, biguanides, α glucosidase inhibitors and insulin sensitizers. Specific examples thereof include insulin and the like for an insulin preparation; glibenclamide, tolbutamide, glyclopyramide, acetohexamide, glimepiride, tolazamide, gliclazide and the like for a sulfonylurea; glybuzole and the like for a sulfonamide; metformin hydrochloride, buformin hydrochloride and the like for biguanides; voglibose, acarbose and the like for an α glucosidase inhibitor; pioglitazone hydrochloride and the like for an insulin sensitizer; and nateglinide and the like for an insulin secretagogue. One or more drugs therefrom can be combined.

In addition, when a compound having a DGAT inhibitory activity (e.g., DGAT1 inhibitory activity) is used as a therapeutic agent for hypertension, besides those mentioned above, it can be used in combination with other therapeutic agents for hypertension. By the other therapeutic agents for hypertension is meant compounds other than a compound having a DGAT inhibitory activity (e.g., DGAT1 inhibitory activity), which are generally used as therapeutic agents for hypertension. Examples thereof include a loop diuretic, an angiotensin converting enzyme inhibitor, an angiotensin II receptor antagonist, a Ca antagonist, a β blocker, an α,β blocker and an α blocker. Specifically, a furosemide sustained-release preparation, captopril, a captopril sustained-release preparation, enalapril maleate, alacepril, delapril hydrochloride, cilazapril, lisinopril, banazepril hydrochloride, imidapril hydrochloride, temocapril hydrochloride, quinapril hydrochloride, trandrapril, perindopril erbumine, losartan potassium, candesartan cilexetil, nicardipine hydrochloride, a nicardipine hydrochloride sustained-release preparation, nilvadipine, nifedipine, a nifedipine sustained-release preparation, benidipine hydrochloride, diltiazem hydrochloride, a diltiazem hydrochloride sustained-release preparation, nisoldipine, nitrendipine, manidipine hydrochloride, barnidipine hydrochloride, efonidipine hydrochloride, amlodipine besylate, felodipine, cilnidipine, aranidipine, propranolol hydrochloride, a propranolol hydrochloride sustained-release preparation, pindolol, a pindolol sustained-release preparation, indenolol hydrochloride, carteolol hydrochloride, a carteolol hydrochloride sustained-release preparation, bunitrolol hydrochloride, a bunitrolol hydrochloride sustained-release preparation, atenolol, acebutolol hydrochloride, metoprolol tartrate, a metoprolol tartrate sustained-release preparation, nipradilol, penbutolol sulfate, tilisolol hydrochloride, carvedilol, bisoprolol fumarate, betaxolol hydrochloride, celiprolol hydrochloride, bopindolol malonate, bevantolol hydrochloride, labetalol hydrochloride, arotinolol hydrochloride, amosulalol hydrochloride, prazosin hydrochloride, terazosin hydrochloride, doxazosin mesylate, bunazosin hydrochloride, a bunazosin hydrochloride sustained-release preparation, urapidil, phentolamine mesylate, and the like can be mentioned. One or more drugs therefrom can be combined.

When a compound having a DGAT inhibitory activity (e.g., DGAT1 inhibitory activity) is used as a therapeutic agent for arteriosclerosis, besides the above-mentioned, it can be used in combination with other therapeutic agents for arteriosclerosist, and when a compound having a DGAT inhibitory activity (e.g., DGAT1 inhibitory activity) is used as a therapeutic agent for coronary diseases, it can be used in combination with other therapeutic agents for coronary diseases.

In this case, the timing of the administration of a drug to be concurrently used with a compound having a DGAT inhibitory activity (e.g., DGAT1 inhibitory activity) is not limited, and they may be administered simultaneously or may be administered in a staggered manner. Moreover, a compound having a DGAT inhibitory activity and a drug to be concurrently used therewith may be prepared as separate pharmaceutical preparation or a single preparation.

### EXAMPLES

Now, one embodiment of the production method of a compound having a DGAT inhibitory activity (e.g., DGAT1 inhibitory activity) is explained in the following, but the production method of the present invention is not limited to these examples.

When the reaction to be mentioned below is carried out, functional groups at positions other than the reaction site may be protected beforehand as necessary and may be deprotected at a suitable stage.

The amount of the solvent to be used for each step is not particularly limited as long as a reaction mixture can be stirred.

As the reagent to be used for each step, its hydrate, salt and the like can be also used as long as the object reaction is not inhibited.

Moreover, the reaction in each step may be carried out according to a conventional method, wherein isolation and purification are performed by appropriately selecting or combining conventional methods, such as crystallization, recrystallization, column chromatography, preparative HPLC and the like.

### Reference Example 1

Step A. Phenyl maleic acid anhydride (20 g, 0.115 mol) was added to a solution of hydrazine monohydrochloride (15.7 g, 0.230 mol) in 80% aqueous EtOH solution (40 mL). The reaction mixture was heated under reflux for 20 hr. This solution was cooled to 0° C. and the obtained precipitate was collected by filtration in vacuo and washed with cooled EtOH (100 mL) to give 4-phenylpyridazine-3,6-diol as a white solid.

¹H NMR (DMSO-d₆): 7.17(s,1H), 7.43(m,5H).

MS(ESI+)m/e=189.1(M+H).

Step B. 4-Phenylpyridazine-3,6-diol (19 g) was added to POCl₃ (50 mL). The reaction mixture was heated under reflux for 4 hr and added dropwise to iced water (300 mL). The obtained precipitate was collected by filtration in vacuo to give 3,6-dichloro-4-phenylpyridazine.

¹H NMR (CDCl₃): 7.48-7.55(m,6H).

MS(ESI+)m/e=225.0(M+H).

Step C. 3,6-Dichloro-4-phenylpyridazine (9.0 g) was added to a solution of diisopropylethylamine (9.39 mL, 53.9 mmol) in dioxane (200 mL). Thereto was added morpholine (3.60 mL, 41.3 mmol) and the reaction mixture was heated under reflux for 18 hr. The solvent was removed in vacuo and replaced by EtOAc (600 mL). This solution was washed with water and brine, dried (MgSO₄), filtered and concentrated in vacuo to give 4-(6-chloro-5-phenylpyridazin-3-yl)morpholine.

¹H NMR (DMSO-d₆): 3.60(m,4H), 3.71(m,4H), 7.34(s,1H), 7.54(m,5H).

MS(ESI+)m/e=276.1(M+H).

Step D. 4-(6-Chloro-5-phenylpyridazin-3-yl)morpholine (9.91 g, 35.9 mmol), HCO₂NH₄ (22.7 g, 0.359 mol) and 10% Pd/C (2 g) were heated in MeOH (200 mL) at 48° C. for 16 hr. The reaction mixture was filtered using celite and the filtrate was concentrated in vacuo to give a yellow solid. This solid was dissolved in CH₂Cl₂, washed with water, dried over MgSO₄, filtered and concentrated in vacuo to give a yellow solid. 4-(5-Phenylpyridazin-3-yl)morpholine was obtained by recrystallization from EtOAc/hexane.

¹H NMR (DMSO-d₆): 3.75(s,8H), 7.64(m,3H), 8.01(m, 2H),

8.23(s,1H), 9.63(s,1H).

MS(ESI+)m/e=242.2(M+H).

Step E. A solution of 4-(5-phenylpyridazin-3-yl)morpholine (200 mg, 0.829 mmol) and 4-bromo-1-butene (252 μL, 2.49 mmol) in CH₃CN (30 mL) was heated under reflux for 12 hr. The volume of the solvent was reduced to 5 mL in vacuo and Et₂O (25 mL) was added. The obtained precipitate was collected by filtration in vacuo and washed with Et₂O to give 1-buta-3-enyl-3-morpholin-4-yl-5-phenylpyridazin-1-ium bromide.

¹H NMR (CDCl₃): 2.71(m,2H), 3.83(m,4H), 3.88(m,4H), 4.86(t,J=6.7 Hz,2H), 4.99(dd,J=0.7, 17.1 Hz,1H), 5.14(dd,J=0.7, 10.3 Hz,1H), 5.88(m,1H), 7.45(m,3H), 8.13(s,1H), 8.18(m,2H), 10.17(s,1H).

MS(ESI+)m/e=296.1(M-Br).

Step F. A solution of diethyl acetylene dicarboxylate (200 μL, 1.24 mmol) and 1M TBAF in THF (912 μL, 0.912 mmol) was added to a solution of 1-buta-3-enyl-3-morpholin-4-yl-5-phenylpyridazin-1-ium bromide (312 mg, 0.829 mmol) in THF (30 mL) and EtOH (5 mL). The reaction mixture was heated under reflux for 12 hr. The solvent was removed in vacuo and the obtained oil was purified by flash column chromatography (silica gel, 10% EtOAc/hexane). The obtained residue was crushed in 1:1 Et₂O/hexane and the precipitate was collected by filtration in vacuo and washed with hexane to give diethyl 7-allyl-2-morpholin-4-yl-4-phenylpyrrolo[1,2-b]pyridazine-5,6-dicarboxylate as an eggshell white solid.

¹H NMR (DMSO-d₆): 0.92(t, J=7.1 Hz, 3H), 1.23(t, J=7.1 Hz, 3H), 3.56(m,6H), 3.74(m,4H), 3.95(d,J=6.4 Hz,2H), 4.20(q,J=7.1 Hz,2H), 5.04(dd,J=1.7, 10.0 Hz,1H), 5.11(dd,J=1.7, 17.1 Hz,1H), 5.97(m,1H), 6.82(s,1H), 7.43(m,2H), 7.49(m,3H).

MS(ESI+)m/e=464.1(M+H).

### Example 1

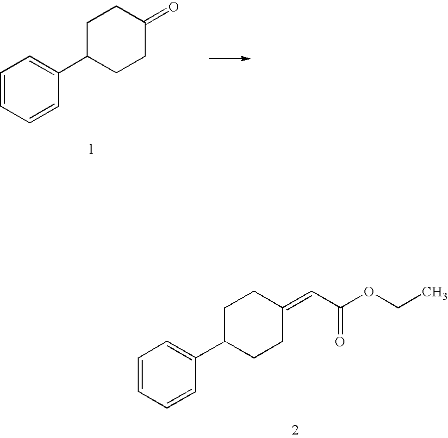

Sodium hydride (60% oil, 517 mg, 12.91 mmol) was added dropwise to a solution of triethyl phosphonoacetate (2.6 mL, 12.91 mmol) in DMF (5.5 mL) at 0° C. The reaction mixture was stirred at room temperature for 30 min. A solution of 4-phenylcyclohexanone (1) in DMF (2.0 mL) was added. After stirring for 0.5 hr, the mixture was poured into 5% aqueous KHSO₄ solution (10 mL) and the mixture was extracted with diethyl ether (10 mL). The organic layer was washed successively with water (5 mL) and brine (5 mL), dried over MgSO₄ and concentrated. The residue was purified by column chromatography (hexane/AcOEt=7/1) to give Compound 2 (2.0 g) as a colorless oil.

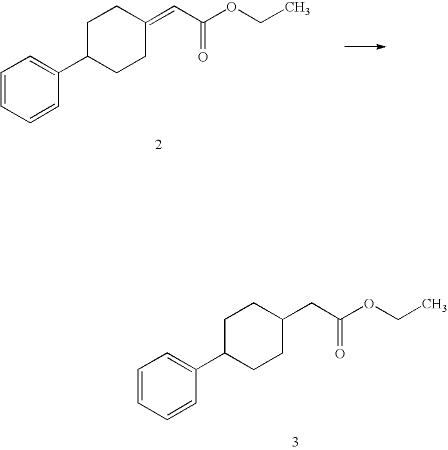

10% Pd/C (50 mg) was added to a stirred solution of Compound 2 (500 mg, 2.05 mmol) in EtOH (5 mL). The mixture was stirred under hydrogen atmosphere at room temperature for 1 hr. The catalyst was removed by filtration and the filtrate was concentrated in vacuo to give crude Product 3 (491 mg) as a colorless oil. This was used in the following step without further purification.

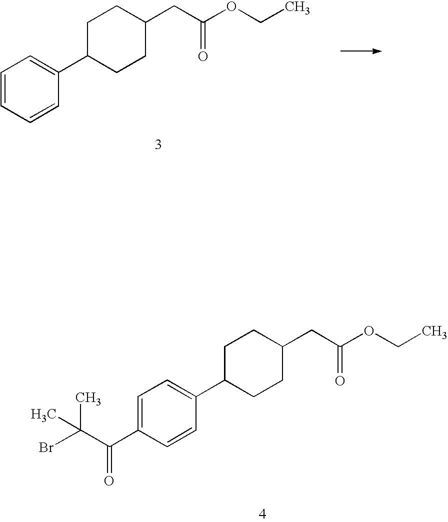

Anhydrous AlCl₃ (440 mg, 3.30 mmol) was added portionwise to a solution of Compound 3 (271 mg, 1.10 mmol) in CH₂Cl₂ (1.4 mL) at 0° C. and then 2-bromoisobutyryl bromide (0.14 mL, 1.10 mmol) was added dropwise. After stirring for 1 hr at 0° C., the mixture was poured into iced water and extracted with CHCl₃ (5 mL). The combined organic layer was washed successively with saturated aqueous NaHCO₃ solution (5 mL) and brine (5 mL), dried over MgSO₄ and concentrated. The residue was purified by column chromatography (hexane/AcOEt=7/1) to give Compound 4 (402 mg) as a colorless oil.

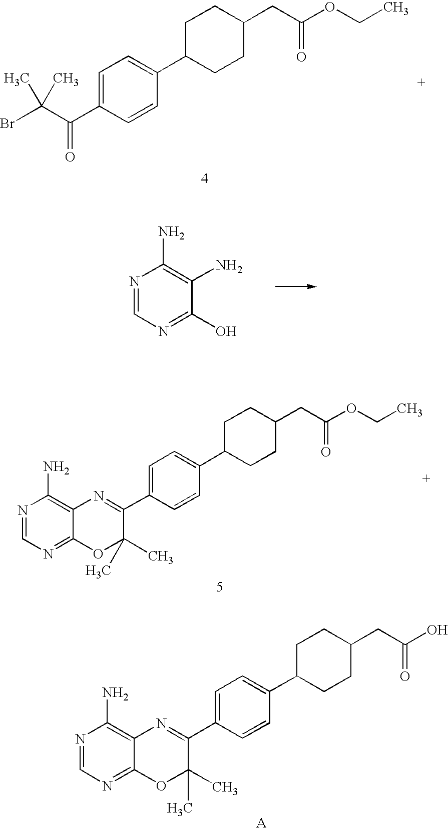

4,5-Diamino-6-hydroxypyrimidine (63.1 mg, 0.50 mmol) was mixed with 1N aqueous HCl solution (0.50 mL, 0.50 mmol), water (2 mL), EtOH (2 mL) and a solution of Compound 4 (395 mg, 1.00 mmol) in EtOH (2 mL). The reaction mixture was refluxed (105° C.) for 12 hr. The reaction mixture was concentrated to a half volume. The residue was adjusted to pH 9-10 with 2N aqueous NaOH solution. The resulting mixture was extracted with AcOEt (5 mL). The aqueous layer was adjusted to pH 3-4 with 10% aqueous citric acid solution and extracted with AcOEt (5 mL). The organic layer was washed with water (5 mL) and brine (5 mL) and dried over MgSO₄. Evaporation of the solvent gave crude Compound A (54 mg, mixture of cis and trans isomers). The first organic layer was washed with water (5 mL) and brine (5 mL) and dried over MgSO₄. Evaporation of the solvent gave crude Compound 5 (126 mg, mixture of cis and trans isomers), which was used for the next step without further purification. To a solution of crude Compound 5 in EtOH (1.2 mL), THF (1.8 mL) and water (1.2 mL) was added 2N aqueous NaOH solution (0.45 mL) after stirring at 40° C. for 4 hr. The reaction mixture was concentrated to a half volume, and added to water (2 mL) and washed with AcOEt (2 mL). The aqueous layer was adjusted to pH 3-4 with 10% aqueous citric acid solution, and extracted with AcOEt (5 mL). The organic layer was washed with water (5 mL) and brine (5 mL) and dried over MgSO₄. Evaporation of the solvent gave a white solid (113 mg). The white solid (113 mg) and crude Compound A (54 mg) were combined and recrystallized from EtOH to give Compound A (92 mg, trans isomer) as white crystals.

m.p.: >270° C.

IR (cm⁻¹): 3320, 2929, 1702, 1601.

MS (ESI+): 395 (100).

¹H NMR (DMSO-d₆, 300 MHz): 1.10-1.16 (m, 2H), 1.45-1.84 (m, 13H), 2.15 (d, 2H, J=6.0 Hz), 2.54 (m, 1H), 6.97 (br s, 2H), 7.30 (d, 2H, J=8.4 Hz), 7.64 (d, 2H, J=8.4 Hz), 7.94 (s, 1H), 11.95 (br s, 1H).

### Example 2

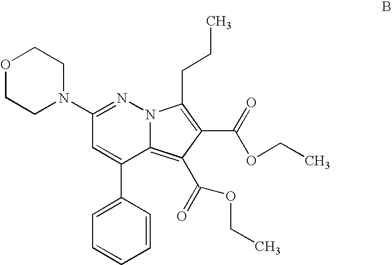

1-Butyl-3-morpholin-4-yl-5-phenylpyridazin-1-ium iodide was prepared from 4-(5-phenylpyridazin-3-yl)morpholine and 1-iodo-butane as described in Step E of Reference Example 1.

¹H NMR (DMSO-d₆): 1.11(t,3H,J=7.4 Hz), 1.52-1.56(m,2H), 2.13-2.19(m,2H), 3.94(s,8H), 4.74(t,2H,J=7.4 Hz), 7.80-7.82(m,3H), 8.17-8.20(m,2H), 8.40(s,1H), 9.80(s,1H).

Diethyl 2-morpholin-4-yl-4-phenyl-7-propylpyrrolo[1,2-b]pyridazine-5,6-dicarboxylate (B) was prepared as described in Step F of Reference Example 1.

¹H NMR (DMSO-d₆): 1.05-1.10(m,6H), 1.39(t,3H,J=7.0 Hz), 1.82-1.88(m,2H), 3.33(t,2H,J=7.8 Hz), 3.67-3.74(m,6H), 3.88-3.91(m,4H), 4.35(q,2H,J=7.0 Hz), 6.94(s,1H), 7.56-7.59(m,2H), 7.62-7.65(m,3H).

MS(ES+)m/e=488.2(M+23).

The Compounds C—P in Table 2 were obtained by a method similar to Example 1, a method disclosed in WO2004/47755, or a method similar thereto. 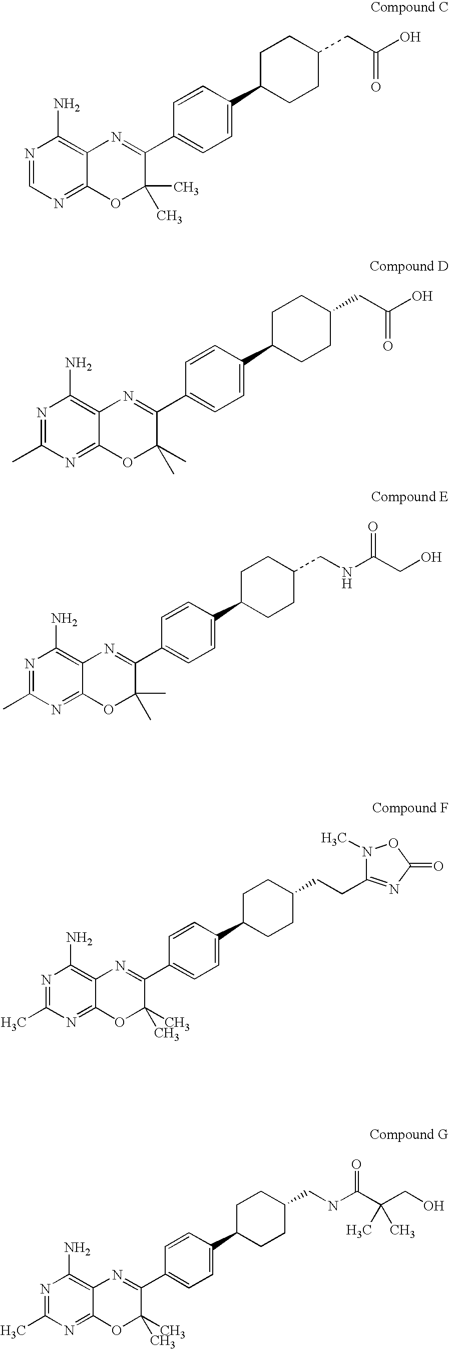 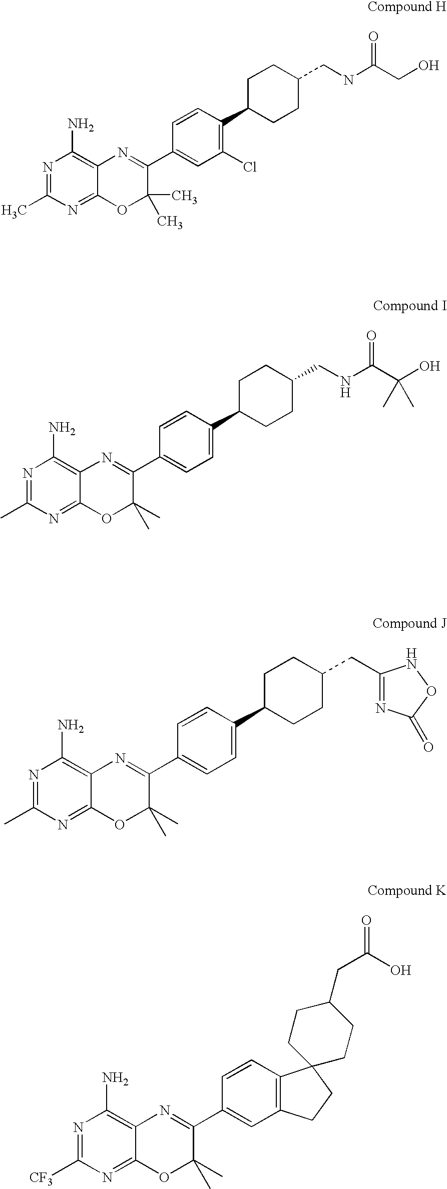 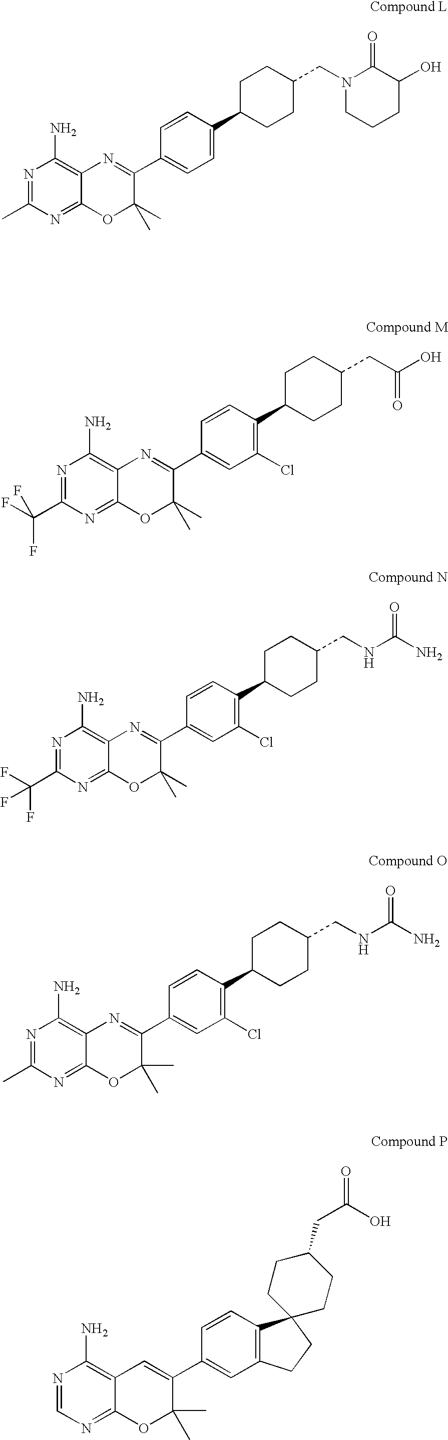
Pharmacological Test

### Experimental Example (1)

Evaluation of Compounds' Effect on Food Consumption in Rats

SD(IGS) rats (Charles River Japan, Inc., 5-10 weeks of age, male) were used for the pharmacological tests. Rats were individually housed in the room set to a light cycle of lights-out at 10 am and lights-on at 10 pm, and were adapted to high fat diet (35% w/w). They were acclimated for more than 10 days prior to experiments under these conditions.

Based on the food consumption during acclimation and the body weight on the day of experiment, rats were grouped with no difference between groups.

As the test compound, Compounds A-P were used. These test compounds were suspended in 0.5% methyl cellulose solution.

Rats were fasted for about 24 hours before experiment. Each dose of the test compound was orally administered just after the lights-out, and immediately thereafter, the feeding of the high fat diet was resumed. The food weight was measured at 1, 4 and 8 hours after the resumption of the feeding to obtain the cumulative food consumption. The inhibitory rate on food consumption was determined by the following formula using the weight of the cumulative food consumption in each group. The test results are shown in Table 1 and 2.

The inhibitory rate on food consumption (%)=(1-test compound group/vehicle group)×100

TABLE 1

| Compound | 1 h | 4 h | 8 h |
|---|---|---|---|
| A (10 mg/kg) | 29 | 37 | 30 |
| B (10 mg/kg) | 26 | 14 | 4 |

TABLE 2

| Compound | | | |
|---|---|---|---|
| (1 mg/kg) | 1 h | 4 h | 8 h |
| C | 24 | 30 | 19 |
| D | 50 | 27 | 17 |
| E | 6 | 15 | 20 |
| F | 11 | 20 | 13 |
| G | 29 | 19 | 15 |
| H | 36 | 36 | 20 |
| I | 30 | 1 | 4 |
| J | 8 | 23 | 10 |
| K | 19 | 37 | 22 |
| L | 10 | 16 | 9 |
| M | 26 | 12 | 14 |
| N | 17 | 6 | 7 |
| O | 20 | 13 | 12 |
| P | 20 | 36 | 28 |

### Experimental Example (2)

### Assay of DGAT1 Enzyme Inhibitory Activity

The enzyme source used for the assay was prepared using a human DGAT1 cDNA isolated from the human liver cDNA library. To be specific, a restriction enzyme cleaving sequence and a flag-labeling sequence were added onto the 5′ side and a restriction enzyme cleaving sequence was added onto the 3′ side of the human DGAT1 cDNA by PCR, and human flag tag DGAT1 baculovirus was prepared using Bac-to-Bac Baculovirus Expression System (Invitrogen). Sf21 insect cells were infected for 24-72 hours, recovered and ruptured in a homogenizing buffer using a microfluidizer. The homogenate was centrifuged at 45,000 rpm for 1 hour, and the cell membrane fractions were recovered and used as an enzyme source.

The DGAT inhibitory activity was measured by Scintillation Proximity Assay (SPA). Human DGAT1 membrane fraction (0.25 μg/well) was mixed with various concentrations of the compound and 200 μM of dioleoyl glycerol (enzyme substrate). 25 μM ¹⁴C decanoyl CoA (radioactive substrate) was added to start the enzyme reaction, and incubated at room temperature for 10 min. Wheat Germ Agglutinin (WGA) SPA beads-suspended 6 mM HgCl₂ (25 μL) was added to terminate the reaction, and the reaction mixture was kept at room temperature for 2 hours to allow adhesion of produced TG onto the SPA beads together with the cell membrane. The mixture was centrifuged at 2,500 rpm for 5 min to precipitate the SPA beads. The radioactivity was measured using Top Count. The test results are shown in Table 3.

TABLE 3

| | |
|---|---|
| Compound A | +++ |
| Compound B | + |
| Compound C | +++ |
| Compound D | +++ |
| Compound E | ++ |
| Compound F | ++ |
| Compound G | ++ |
| Compound H | ++ |
| Compound I | ++ |
| Compound J | +++ |
| Compound K | +++ |
| Compound L | ++ |
| Compound M | +++ |
| Compound N | ++ |
| Compound O | ++ |
| Compound P | +++ |

+: IC₅₀ ≧ 0.1 μM
++: 0.1 μM > IC₅₀ ≧ 0.01 μM
+++: 0.01 μM > IC₅₀

### INDUSTRIAL APPLICABILITY

A compound having a DGAT inhibitory activity (e.g., DGAT1 inhibitory activity), a prodrug thereof and pharmaceutically acceptable salts thereof are useful as anorectics. Besides the anorectic, they are useful as drugs for treating or preventing obesity, hyperlipidemia, diabetes, arteriosclerosis, coronary disease and hypertension.

Moreover, a compound having a DGAT inhibitory activity (e.g., DGAT1 inhibitory activity) is useful for combination therapy with other therapeutic agents for obesity, therapeutic agents for arteriosclerosis, therapeutic agents for coronary diseases, therapeutic agents for hypertension, therapeutic agents for diabetes or therapeutic agents for hyperlipidemia.

This application is based on patent application No. 24812/2004 filed in Japan and patent provisional application No. 60/598,037 in the United States of America, the contents of which are hereby incorporated by reference.

1. An anorectic comprising, as an active ingredient, a compound having a DGAT (diacylglycerol acyltransferase) inhibitory activity or a prodrug thereof or a pharmaceutically acceptable salt thereof.

2. The anorectic of claim 1, wherein the compound having a DGAT inhibitory activity is a compound represented by the following formula (1): 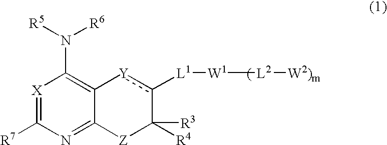
wherein
X is C(R¹) or N,
wherein R¹ is a hydrogen atom, a C₁₋₈ alkyl group, a C₂₋₈ alkenyl group, a C₂₋₈ alkynyl group, a C₁₋₈ fluoroalkyl group, an aryl group, an aryl C₁₋₄ alkyl group, C(O)R^{a}, CO₂R^{a} or C(O)NR^{a}R^{b}, wherein R^{a} and R^{b} are the same or different and each is a hydrogen atom, a C₁₋₈ alkyl group, a C₂₋₈ alkenyl group, a C₂₋₈ alkynyl group, a C₁₋₈ fluoroalkyl group, an aryl group or an aryl C₁₋₄ alkyl group;
Y is C(R¹), C(R²)(R²), N or N(R²),
wherein R¹ is as defined above and each R² is independently a hydrogen atom, a C₁₋₈ alkyl group, a C₂₋₈ alkenyl group, a C₂₋₈ alkynyl group, a C₁₋₈ fluoroalkyl group, C(O)R^{a}, CO₂R^{a}, C(O)NR^{a}R^{b}, an aryl group or an aryl C₁₋₄ alkyl group, wherein R^{a} and R^{b} are as defined above;
Z is O or S;
W¹ is an optionally substituted C₃₋₈ cycloalkylene group, an optionally substituted C₃₋₈ heterocycloalkylene group, an optionally substituted arylene group or an optionally substituted heteroarylene group;
W² is an optionally substituted C₃₋₈ cycloalkyl group, an optionally substituted C₃₋₈ heterocycloalkyl group, an optionally substituted aryl group or an optionally substituted heteroaryl group;
L¹ is a single bond, a C₁₋₄ alkylene group, a C₂₋₄ alkenylene group, O, C(O)N(R^{a}) or N(R^{a})C(O), wherein R^{a} is as defined above;
L² is a single bond, 0, a C₁₋₄ alkylene group, a C₂₋₄ alkenylene group, a C₁₋₄ heteroalkylene group, C(O)N(R^{a}) or N(R^{a})C(O), wherein R^{a} is as defined above;
m is 0 or 1;
when m is 1 and L² is a single bond, a substituent of W² may form, together with a substituent of W¹, a 5 to 7-membered ring that is condensed with W₁ and forms a fused ring or spiro ring with W²;
R³ and R⁴
are the same or different and each is a hydrogen atom, a C₁₋₈ alkyl group, a C₂₋₈ alkenyl group, a C₂₋₈ alkynyl group, C(O)R^{a}, CO₂R^{a}, C(O)NR^{a}R^{b} or a C₁₋₄ alkylene-OR^{a} group, wherein R^{a} and R^{b} are as defined above, or R³ and R⁴ may form a 3 to 6-membered ring together with the carbon atom binding thereto; or
R², R³ or R⁴
may form, together with W¹, a 5 to 7-membered ring optionally having, in the ring, 1 to 3 heteroatoms selected from a nitrogen atom, an oxygen atom and a sulfur atom;
R⁵ and R⁶
are the same or different and each is a hydrogen atom, a C₁₋₈ alkyl group, a C₂₋₈ alkenyl group, a C₂₋₈ alkynyl group, C(O)R^{a} or CO₂R^{a}, wherein R^{a} is as defined above, R⁵ and R⁶ may form an N-containing 5 to 7-membered ring together with the nitrogen atom binding thereto, or, when X is C(R¹), R⁵ or R⁶ may form, together with R¹, an N-containing 5 to 7-membered ring, wherein N may be substituted by R⁵ or R⁶;
R⁷ is a hydrogen atom, a C₁₋₈ alkyl group, a C₁₋₄ haloalkyl group, a C₂₋₈ alkenyl group, a C₂₋₈ alkynyl group, C(O)R^{a}, OR^{a} or NR^{a}R^{b}, wherein R^{a} and R^{b} are as defined above, or, when X is C(R¹), R⁷ may form, together with R¹, a 5 to 7-membered ring; and [symbol] is a single bond or a double bond;
provided that the following compound is excluded: 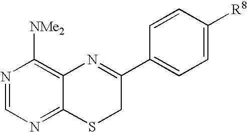
wherein R⁸ is a hydrogen atom, a nitro group, a chlorine atom, a methoxy group, a methyl group or a phenyl group.

3. The anorectic of claim 1, wherein the compound having a DGAT inhibitory activity is a compound represented by the following formula (2): 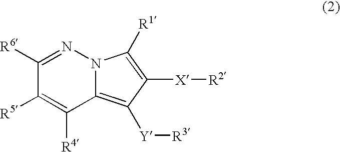
wherein
X′ and Y′
are the same or different and each is a single bond, a C₁₋₄ alkylene group, a C₂₋₄ heteroalkylene group, —O—, —CO₂—, —S(O)ₖ—,
—C(O)—, —NR⁷′—, —C(O)NR⁷′—, —N(R⁸′)C(O)NR⁷′—,
—N(R⁷′)CO₂—, —SO₂NR⁷′—, —N(R⁸′)SO₂NR⁷′—, —NR⁷′C(O)—, —O—C(O)N(R⁷′)- or —NR⁷′SO₂—,
wherein R⁷′ and R⁸′ are the same or different and each is a hydrogen atom, a C₁₋₈ alkyl group, an aryl group or an aryl C₁₋₄ alkyl group and k is an integer of 0 or 1-2;
R¹′ is a hydrogen atom, a halogen atom, a C¹⁻⁸ alkyl group, a C₂₋₈ alkenyl group, a C₂₋₈ alkynyl group, a C₁₋₈ fluoroalkyl group, a C₃₋₈ cycloalkyl group, a C₂₋₈ heteroalkyl group, a C₂₋₈ heteroalkenyl group, a C₃₋₈ heterocycloalkyl group, an aryl group, an aryl C₁₋₄ alkyl group, a heteroaryl group, OR^{a}′, SR^{a}′, C(O)R^{a}′, CO₂R^{a}′, C(O)NR^{a}′R^{b}′, SO₂R^{a}′, SO₂NR^{a}′R^{b}′, a nitro group or a cyano group,
wherein R^{a}′ and R^{b}′ are the same or different and each is a hydrogen atom, a C₁₋₈ alkyl group, a C₃₋₈ cycloalkyl group, an aryl group or an aryl C₁₋₄ alkyl group;
R²′ is a C₁₋₈ alkyl group, an aryl C₁₋₄ alkyl group, OR^{a}′, a halogen atom, a nitro group, NR^{a}′R^{b}′, a cyano group or W¹′, wherein W¹′ is 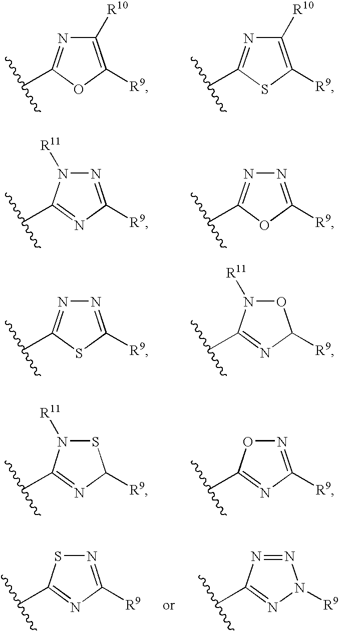
wherein R⁹ and R¹⁰ are the same or different and each is a hydrogen atom, a C₁₋₈ alkyl group, a C₂₋₈ alkenyl group, a C₂₋₈ alkynyl group, a C₁₋₈ fluoroalkyl group, an aryl group or an aryl C₁₋₄ alkyl group, or R⁹ and R¹⁰ may be linked to form a 5 to 7-membered ring optionally having, in the ring, 1 to 3 heteroatoms selected from a nitrogen atom, an oxygen atom and a sulfur atom, R¹¹ is a hydrogen atom, a C₁₋₈ alkyl group, an aryl group or an aryl C₁₋₄ alkyl group, and R^{a}′ and R^{b}′ are as defined above; or
R¹′ and R²′
may be linked to form a 5 to 7-membered ring optionally having, in the ring, one heteroatom selected from a nitrogen atom, an oxygen atom and a sulfur atom;
R³′ is a hydrogen atom, a C₁₋₈ alkyl group, an aryl C₁₋₄ alkyl group, OR^{a}′, a halogen atom, a nitro group, NR^{a}′R^{b}′, a cyano group or W²′, wherein W²′ is 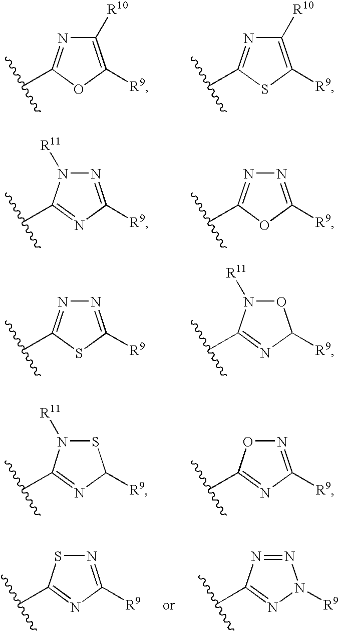
wherein R⁹, R¹⁰ and R¹¹ are as defined above, and R^{a}′ and R^{b}′ are as defined above; or
R²′ and R³′
may be linked to form a 5 to 7-membered ring optionally having, in the ring, one heteroatom selected from a nitrogen atom, an oxygen atom and a sulfur atom;
R⁴′ is a C₁₋₈ alkyl group, a C₂₋₈ alkenyl group, a C₂₋₈ alkynyl group, a C₁₋₄ fluoroalkyl group, a C₂₋₈ heteroalkyl group, a C₂₋₈ heteroalkenyl group, a C₃₋₈ cycloalkyl group, a C₃₋₈ heterocycloalkyl group, an aryl group, an aryl C₁₋₄ alkyl group, a heteroaryl group, OR^{a}′, SR^{a}′, NR^{a}′R^{b}′, C(O)R^{a}′, CO₂R^{a}′, C(O)NR^{a}′R^{b}′, SO₂R^{a}′ or SO₂NR^{a}′R^{b}′, wherein R^{a}′ and R^{b}′ are as defined above;
R⁵′ is a hydrogen atom, a C₁₋₈ alkyl group, a C¹⁻⁸ fluoroalkyl group, a C₃₋₈ cycloalkyl group, a C₂₋₈ heteroalkyl group, a C₂₋₈ heteroalkenyl group, a C₃₋₈ heterocycloalkyl group, an aryl group, an aryl C₁₋₄ alkyl group, a heteroaryl group, a halogen atom, OR^{a}′, NR^{a}′R^{b}′, a cyano group, C(O)R^{a}′, CO₂R^{a}′, C(O)NR^{a}′R^{b}′, OC(O)R^{a}′, OCO₂R^{c}, OC(O)NR^{a}′R^{b}′, NR^{a}′C(O)R^{b}′NR^{a}′, CO₂R^{c} or NR^{a}′C(O)NR^{a}′R^{b}′, wherein R^{a}′ and R^{b}′ are as defined above and R^{c} is a C₁₋₈ alkyl group, a C₃₋₈ cycloalkyl group, an aryl group or an aryl C₁₋₄ alkyl group; and
R⁶′ is OR^{d}, NR^{d}R^{e} or S(O)_{m′}R^{d},
wherein R^{d} and R^{e} are the same or different and each is a hydrogen atom, a C₁₋₈ alkyl group, a C₂₋₈ alkenyl group, a C₂₋₈ alkynyl group, a C₁₋₈ fluoroalkyl group, C(O)R^{f}, an aryl group or an aryl C₁₋₄ alkyl group, m′ is an integer of 0 or 1-2, wherein R^{f} is a hydrogen atom, a C₁₋₈ alkyl group, an amino group, a C₁₋₄ alkylamino group, a di(C₁₋₄ alkyl)amino group, an aryl C₁₋₄ alkyl group or a C₁₋₈ alkoxy group, or when R⁶′ is NR^{d}R^{e}, R^{d} and R^{e} may form, together with the nitrogen atom binding thereto, an N-containing 4 to 7-membered heterocyclic ring wherein the ring may further contain 1 or 2 heteroatoms selected from a nitrogen atom, an oxygen atom and a sulfur atom.

4. The anorectic of claim 1, wherein the compound having a DGAT inhibitory activity is a compound represented by the following formula (3): 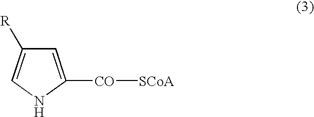
wherein R is a C₅₋₂₅ alkyl group or a C₅₋₂₅ alkenyl group, and ScoA shows a residue which is a coenzyme A deficient in the hydrogen atom of a terminal mercapto group.

5. The anorectic of claim 1, wherein the compound having a DGAT inhibitory activity is a compound represented by the following formula (4): 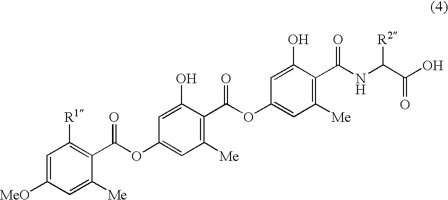
wherein, when R¹″ is a hydrogen atom, R²″ is a methyl group or an isopropyl group, and when R¹″ is a methyl group, R²″ is a methyl group.

6. The anorectic of claim 1, wherein the compound having a DGAT inhibitory activity is a compound represented by the following formula (5): 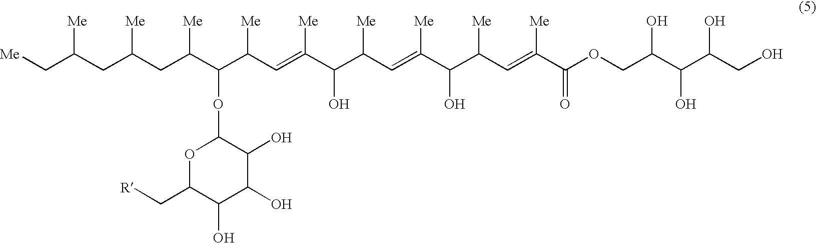
wherein R′ is a hydroxyl group or an acetyloxy group.

7. The anorectic of claim 1, wherein the compound having a DGAT inhibitory activity is a compound represented by the following formula (6): 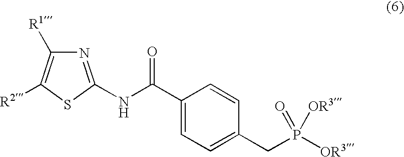
wherein R¹′″ is a phenyl group or a halogen-substituted phenyl group, R²′″ is a hydrogen atom, a C₁₋₆ alkyl group, a carboxyl group, a C₁₋₆ alkoxy-carbonyl group, a cyano group, a C₁₋₆ alkyl-carbamoyl group, a N,N-di(C₁₋₆ alkyl)-carbamoyl group or a pyrrolidinocarbonyl group, and R³′″ is a C₁₋₆ alkyl group.

8. A method for suppressing appetite, which comprises administering a pharmaceutically effective amount of an anorectic of claim 1 to a mammal.

9. A method for treating or preventing obesity, which comprises administering a pharmaceutically effective amount of an anorectic of claim 1 to a mammal.

10. A method for treating or preventing hyperlipidemia, which comprises administering a pharmaceutically effective amount of an anorectic of claim 1 to a mammal.

11. A method for treating or preventing diabetes, which comprises administering a pharmaceutically effective amount of an anorectic of claim 1 to a mammal.

12. A method for treating or preventing arteriosclerosis, which comprises administering a pharmaceutically effective amount of an anorectic of claim 1 to a mammal.

13. A method for treating or preventing a coronary disease, which comprises administering a pharmaceutically effective amount of an anorectic of claim 1 to a mammal.

14. A method for treating or preventing hypertension, which comprises administering a pharmaceutically effective amount of an anorectic of claim 1 to a mammal.

15. The method of claim 9, which further comprises administering a pharmaceutically effective amount of other therapeutic agent for obesity to a mammal.

16. The method of claim 15, wherein said other therapeutic agent for obesity is one or more drugs selected from the group consisting of mazindol, orlistat and sibutramine.

17. The method of claim 10, which further comprises administering a pharmaceutically effective amount of other therapeutic agent for hyperlipidemia to a mammal.

18. The method of claim 17, wherein said other therapeutic agent for hyperlipidemia is a statin drug.

19. The method of claim 18, wherein the statin drug is one or more drugs selected from the group consisting of lovastatin, simvastatin, pravastatin, fluvastatin, atorvastatin, cerivastatin, pitavastatin, nisvastatin and rosuvastatin.

20. The method of claim 17, wherein said other therapeutic agent for hyperlipidemia is a fibrate drug.

21. The method of claim 20, wherein the fibrate drug is one or more drugs selected from the group consisting of clofibrate, clinofibrate, sinfibrate, fenofibrate, bezafibrate and gemfibrozil.

22. The method of claim 17, wherein said other therapeutic agent for hyperlipidemia is probucol.

23. The method of claim 17, wherein said other therapeutic agent for hyperlipidemia is nicotinic acid.

24. The method of claim 17, wherein said other therapeutic agent for hyperlipidemia is a cholesterol absorption suppressant.

25. The method of claim 24, wherein the cholesterol absorption suppressant is one or more drugs selected from the group consisting of ezetimibe, colestimide, colestyramine and colestipol.

26. The method of claim 17, wherein said other therapeutic agent for hyperlipidemia is one or more drugs selected from the group consisting of an MTP inhibitor, an ACAT inhibitor and a CETP inhibitor.

27. The method of claim 11, which further comprises administering a pharmaceutically effective amount of other therapeutic agent for diabetes to a mammal.

28. The method of claim 27, wherein said other therapeutic agent for diabetes is one or more drugs selected from the group consisting of an insulin preparation, a sulfonylurea, an insulin secretagogue, a sulfonamide, a biguanide, an α glucosidase inhibitor and an insulin sensitizer.

29. The method of claim 27, wherein said other therapeutic agent for diabetes is one or more drugs selected from the group consisting of insulin, glibenclamide, tolbutamide, glyclopyramide, acetohexamide, glimepiride, tolazamide, gliclazide, nateglinide, glybuzole, metformin hydrochloride, buformin hydrochloride, voglibose, acarbose and pioglitazone hydrochloride.

30. The method of claim 12, which further comprises administering a pharmaceutically effective amount of other therapeutic agent for arteriosclerosis to a mammal.

31. The method of claim 13, which further comprises administering a pharmaceutically effective amount of other therapeutic agent for coronary diseases to a mammal.

32. The method of claim 14, which further comprises administering a pharmaceutically effective amount of other therapeutic agent for hypertension to a mammal.

33. The method of claim 32, wherein said other therapeutic agent for hypertension is one or more drugs selected from the group consisting of a loop diuretic, an angiotensin converting enzyme inhibitor, an angiotensin II receptor antagonist, a Ca antagonist, a β blocker, an α,β blocker and an a blocker.

34. The method of claim 32, wherein said other therapeutic agent for hypertension is one or more drugs selected from the group consisting of a furosemide sustained-release preparation, captopril, a captopril sustained-release preparation, enalapril maleate, alacepril, delapril hydrochloride, cilazapril, lisinopril, banazepril hydrochloride, imidapril hydrochloride, temocapril hydrochloride, quinapril hydrochloride, trandrapril, perindopril erbumine, losartan potassium, candesartan cilexetil, nicardipine hydrochloride, a nicardipine hydrochloride sustained-release preparation, nilvadipine, nifedipine, a nifedipine sustained-release preparation, benidipine hydrochloride, diltiazem hydrochloride, a diltiazem hydrochloride sustained-release preparation, nisoldipine, nitrendipine, manidipine hydrochloride, barnidipine hydrochloride, efonidipine hydrochloride, amlodipine besylate, felodipine, cilnidipine, aranidipine, propranolol hydrochloride, a propranolol hydrochloride sustained-release preparation, pindolol, a pindolol sustained-release preparation, indenolol hydrochloride, carteolol hydrochloride, a carteolol hydrochloride sustained-release preparation, bunitrolol hydrochloride, a bunitrolol hydrochloride sustained-release preparation, atenolol, acebutolol hydrochloride, metoprolol tartrate, a metoprolol tartrate sustained-release preparation, nipradilol, penbutolol sulfate, tilisolol hydrochloride, carvedilol, bisoprolol fumarate, betaxolol hydrochloride, celiprolol hydrochloride, bopindolol malonate, bevantolol hydrochloride, labetalol hydrochloride, arotinolol hydrochloride, amosulalol hydrochloride, prazosin hydrochloride, terazosin hydrochloride, doxazosin mesylate, bunazosin hydrochloride, a bunazosin hydrochloride sustained-release preparation, urapidil and phentolamine mesylate.
